(19) United States
(12) Patent Application Publication
(10) Pub. No.: US 2026/0035467 A1
(43) Pub. Date: Feb. 5, 2026

(54) METHODS FOR TREATING PRIMARY SJOGREN'S SYNDROME USING FCRN ANTAGONISTS

(71) Applicant: argenx BV, Ghent (BE)

(72) Inventors: Joost Johannes van Middendorp, Ghent (BE); Julie Jacobs, Ghent (BE); Despoina Papadopoulou, Ghent (BE); Isabelle Maria A. Peene, Ghent (BE); Dirk Elewaut, Ghent (BE)

(21) Appl. No.: 19/266,255

(22) Filed: Jul. 11, 2025

Related U.S. Application Data
(63) Continuation of application No. PCT/IB2024/000041, filed on Jan. 12, 2024.
(60) Provisional application No. 63/438,667, filed on Jan. 12, 2023.

(51) Int. Cl. C07K 16/28; A61K 39/00; A61P 37/02

(52) U.S. Cl. CPC C07K 16/283; A61P 37/02; A61K 2039/505; A61K 2039/545

(57) ABSTRACT

Provided herein are methods of treating primary Sjögren's syndrome (pSS) using an effective amount of a human neonatal Fc receptor (FcRn) antagonist. FcRn antagonists for use in the treatment of pSS and for use in the manufacture of a medicament for the treatment of pSS are also provided herein.

### CROSS REFERENCE TO RELATED APPLICATIONS

This application is a Continuation of International Patent Application No. PCT/IB2024/000041, filed Jan. 12, 2024, which claims the benefit of and priority to U.S. Provisional Patent Application No. 63/438,667, filed Jan. 12, 2023, the entire disclosure of each of which is hereby incorporated by reference.

### REFERENCE TO SEQUENCE LISTING

This application contains a sequence listing which has been submitted electronically in ST.26 format and is hereby incorporated by reference in its entirety (said ST.26 copy, created on Jul. 9, 2025, is named “404373-T2215US_SL.xml” and is 40,732 bytes in size).

### FIELD

The present disclosure relates to methods of treating primary Sjögren's syndrome (pSS). The methods involve use of an antagonist of human neonatal Fc receptor (FcRn), which in certain embodiments is efgartigimod.

### BACKGROUND

pSS is a chronic, progressive autoimmune disease of unclear etiology, typically presented as an exocrinopathy. Along with symptoms of extensive dryness, manifestations include profound fatigue, chronic pain, extraglandular organ system involvement, and increased risk of lymphomas. A hallmark of pSS is B cell hyperactivity, causing a vicious cycle of immune activation through cytokine production, antigen presentation, and autoantibody secretion, potentially causing tissue damage. Currently, no immunomodulatory treatment is available for pSS.

Currently, the underlying pathophysiology and effective treatments are unknown for pSS. Pharmacologic therapy mainly focuses on symptoms, targeting reducing discomfort associated with dry eyes and mouth and/or stimulation of tear and saliva production.

Accordingly, there is a need in the art for improved pSS treatment options.

Therapeutic antagonism of FcRn, a major histocompatibility complex class I-like molecule that is involved in the recycling of immunoglobulin G (IgG) and is thus responsible for the long half-life of IgG, has been explored as a strategy to treat IgG-mediated autoimmune diseases such as generalized myasthenia gravis (gMG), immune thrombocytopenia (ITP), and pemphigus (pemphigus vulgaris (PV) and pemphigus foliaceus (PF)). The remarkable clinical efficacy of FcRn antagonism appears to be directly linked to early removal of pathogenic IgG autoantibodies from circulation.

Pathogenic autoantibodies and immune complexes are involved in the development and/or progression of pSS. By reducing pathogenic autoantibodies and immune complexes, FcRn antagonists may provide a safer, more effective treatment option for patients with pSS.

### SUMMARY

The instant disclosure is broadly directed to methods for treating pSS with FcRn antagonists.

The instant disclosure provides a method of treating pSS in a subject in need thereof, the method comprising administering to the subject an effective amount of a human neonatal Fc receptor (FcRn) antagonist. In some embodiments, the FcRn antagonist comprises two, three, or four FcRn binding regions. In some embodiments, the FcRn antagonist comprises or consists of a variant Fc region or FcRn binding fragment thereof. In some embodiments, the variant Fc region or FcRn binding fragment thereof binds to FcRn with a higher affinity at pH 6.0 as compared to a corresponding wild-type Fc region. In some embodiments the variant Fc region or FcRn binding fragment thereof binds to FcRn with a higher affinity at pH 7.4 as compared to a corresponding wild-type Fc region.

In some embodiments, the variant Fc region comprises or consists of a first Fc domain and a second Fc domain which form a homodimer or heterodimer. In some embodiments, the first Fc domain and/or the second Fc domain comprise amino acids Y, T, E, K, and F at EU positions 252, 254, 256, 433, and 434, respectively. In some embodiments, the first Fc domain and/or the second Fc domain comprise amino acids Y, T, E, K, F, and Y at EU positions 252, 254, 256, 433, 434, and 436, respectively.

In some embodiments, the first Fc domain and/or the second Fc domain comprise an amino acid sequence independently selected from the group consisting of SEQ ID NO: 2, SEQ ID NO: 3, SEQ ID NO: 20, and SEQ ID NO: 21. In some embodiments, the first Fc domain and the second Fc domain comprise an amino acid sequence independently selected from the group consisting of SEQ ID NO: 2, SEQ ID NO: 3, SEQ ID NO: 20, and SEQ ID NO: 21. In some embodiments, the FcRn antagonist is efgartigimod.

In some embodiments, the FcRn antagonist is an anti-FcRn antibody.

In some embodiments, the FcRn antagonist is administered to the subject at a fixed dose of 20 mg to 20,000 mg or at a dose of 0.2 mg/kg to 200 mg/kg.

In some embodiments, the FcRn antagonist is administered intravenously once weekly or once every two weeks. In some embodiments, the FcRn antagonist is administered intravenously at a dose of from 2 mg/kg to 200 mg/kg once weekly or once every two weeks. In some embodiments, the FcRn antagonist is administered intravenously at a dose of 3 mg/kg to 60 mg/kg once weekly or once every two weeks. In some embodiments, the FcRn antagonist is administered intravenously at a dose of 10 mg/kg to 30 mg/kg once weekly or once every two weeks. In some embodiments, the FcRn antagonist is administered intravenously at a dose of 10 mg/kg once weekly or once every two weeks. In some embodiments, the FcRn antagonist is administered intravenously at a dose of 25 mg/kg once weekly or once every two weeks.

In some embodiments, the FcRn antagonist is administered subcutaneously once weekly, once every two weeks, once every three weeks, once every four weeks, once monthly, or once every six weeks. In some embodiments, the FcRn antagonist is administered subcutaneously at a fixed dose of 200 mg to 20,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly or once every two weeks.

In some embodiments, the FcRn antagonist is administered for 24 weeks or less. In some embodiments, the FcRn antagonist is administered for at least 24 weeks. In some embodiments, the FcRn antagonist is administered for 52 weeks or less. In some embodiments, the FcRn antagonist is administered for at least 52 weeks.

In some embodiments, the method further comprises administering to the subject an effective amount of one or more of a corticosteroid, an antimalarial, a disease-modifying anti-rheumatic drug (DMARD), a janus kinase (JAK) inhibitor, a pharmacological stimulant for salivary and lacrimal glands, an anticholinergic agent, or a topical ophthalmic agent. In some embodiments, the corticosteroid is a systemic corticosteroid. In some embodiments, the corticosteroid is a topical corticosteroid.

In some embodiments, the subject meets the ACR-EULAR classification criteria for pSS. In some embodiments, the subject met the ACR-EULAR classification≤7 years before administration of the FcRn antagonist.

In some embodiments, the subject has at least a moderate level of systemic disease activity. In some embodiments, the subject has a EULAR Sjögren's syndrome disease activity index (ESSDAI) score of ≥5.

In some embodiments, the subject has a detectable serum level of a pSS-related autoantibody. In some embodiments, the subject has a detectable serum level of an anti-Ro/SS-A antibody or an anti-La/SS-B antibody.

In some embodiments, the subject has an unstimulated whole salivary flow (UWSF) rate>0 and/or a stimulated whole salivary flow (SWSF) rate>0.10.

In some embodiments, the subject shows one or more responses following administration of the FcRn antagonist, wherein the responses are selected from the group consisting of: a) a clinical ESSDAI (clinESSDAI) score of <5 points; b) a decrease in EULAR Sjögren's syndrome patient reported index (ESSPRI) score of ≥1 point or ≥15%, compared to a baseline value; c) an increase in tear gland function; d) an increase in salivary gland function; and e) a decrease in serum rheumatoid factor (RF) of at least 25%, compared to a baseline value, or a decrease in serum IgG of at least 10%, compared to a baseline value. In some embodiments, the responses are measured 16 weeks or 24 weeks following administration of the FcRn antagonist.

In some embodiments, the subject shows three or more responses following administration of the FcRn antagonist, wherein the responses are selected from the group consisting of: a) a clinESSDAI score of <5 points; b) a decrease in ESSPRI score of ≥1 point or ≥15%, compared to a baseline value; c) an increase in tear gland function; d) an increase in salivary gland function; and e) a decrease in serum RF of at least 25%, compared to a baseline value, or a decrease in serum IgG of at least 10%, compared to a baseline value. In some embodiments, the responses are measured 16 weeks or 24 weeks following administration of the FcRn antagonist.

In some embodiments, the increase in tear gland function is measured by Schirmer's test and/or ocular staining score (OSS), wherein if the subject shows a baseline value of ≤5 mm as measured by Schirmer's test, a response is defined as an increase of at least 5 mm from the baseline value; or if the subject shows a baseline value of ≥3 points as measured by OSS, a response is defined as a decrease of at least 2 points from the baseline value; or if the subject shows a baseline value of >5 mm as measured by Schirmer's test and shows a baseline value of <3 points as measured by OSS, a response is defined as no change that results in an abnormal OSS or Schirmer's score. In some embodiments, the responses are measured 16 weeks or 24 weeks following administration of the FcRn antagonist.

In some embodiments, the increase in salivary gland function is measured by UWSF and/or salivary gland ultrasonography (SGUS), wherein a response is defined as an increase in UWSF of at least 25%, compared to a baseline value if the baseline value is >0 mL/min, or any increase in UWSF if the baseline value is 0 mL/min; or a decrease in Hocevar score as measured by SGUS of at least 25%, compared to a baseline value. In some embodiments, the responses are measured 16 weeks or 24 weeks following administration of the FcRn antagonist.

In some embodiments, the subject shows a change in CD45+lymphocytic infiltrate in the parotid gland following administration of the FcRn antagonist, compared to a baseline value. In some embodiments, the subject shows a decrease in CD45+lymphocytic infiltrate in the parotid gland following administration of the FcRn antagonist of at least 5%, at least 10%, at least 15%, at least 20%, at least 25%, at least 30%, at least 40%, at least 50%, at least 60%, at least 70%, at least 80%, or at least 90%, compared to a baseline value. In some embodiments, the CD45+lymphocytic infiltrate in the parotid gland is measured 24 weeks following administration of the FcRn antagonist.

In some embodiments, the subject shows a change in B/B+T cell ratio in the parotid gland following administration of the FcRn antagonist, compared to a baseline value. In some embodiments, the subject shows a decrease in B/B+T cell ratio in the parotid gland following administration of the FcRn antagonist of at least 5%, at least 10%, at least 15%, at least 20%, at least 25%, at least 30%, at least 40%, at least 50%, at least 60%, at least 70%, at least 80%, or at least 90%, compared to a baseline value. In some embodiments, the B/B+T cell ratio in the parotid gland is measured 24 weeks following administration of the FcRn antagonist.

In some embodiments, the subject shows a decrease in ESSDAI score, clinESSDAI score, and/or ESSPRI score following administration of the FcRn antagonist, compared to a baseline value. In some embodiments, the ESSDAI score, the clinESSDAI score, and/or the ESSPRI score is measured 16 weeks or 24 weeks following administration of the FcRn antagonist. In some embodiments, the subject shows a decrease of at least 3 points in the ESSDAI score and/or the clinESSDAI score, following administration of the FcRn antagonist. In some embodiments, the subject has an ESSDAI score of <5 and/or a clinESSDAI score of <5, following administration of the FcRn antagonist. In some embodiments, the subject shows a decrease of at least 1 point or a decrease of >15% in the ESSPRI score, following administration of the FcRn antagonist.

In some embodiments, the subject shows an increase in Sjögren's Tool for Assessing Response (STAR) score following administration of the FcRn antagonist, compared to a baseline value. In some embodiments, the STAR score is measured 24 weeks following administration of the FcRn antagonist. In some embodiments, the subject has a STAR score of ≥5 following administration of the FcRn antagonist.

In some embodiments, the subject shows an improvement in total Multidimensional Fatigue Inventory (MFI) score, SF-36 physical component score, SF-36 mental component score, PGA score, EQ-5D-5L score, VAS score, ESSPRI dryness score, ESSPRI fatigue score, ESSPRI pain score, and/or PASS score following administration of the FcRn antagonist, compared to a baseline value. In some embodiments, the total MFI score, the SF-36 physical component score, the SF-36 mental component score, the PGA score, the EQ-5D-5L score, the VAS score, the ESSPRI dryness score, the ESSPRI fatigue score, the ESSPRI pain score, and/or the PASS score is measured at 16 weeks or 24 weeks following administration of the FcRn antagonist.

In some embodiments, the subject shows a change in SWSF rate, UWSF rate, Hocevar score, Schirmer's test score, and/or OSS, following administration of the FcRn antagonist, compared to a baseline value. In some embodiments, the SWSF rate, the UWSF rate, the Hocevar score, the Schirmer's test score, and/or the OSS is measured at 16 weeks or 24 weeks following administration of the FcRn antagonist.

In some embodiments, the subject shows a reduction in a serum level of total IgG, RF, an autoantibody, a cytokine/chemokine, an immune complex, or a marker of complement activation following administration of the FcRn antagonist, compared to a baseline value. In some embodiments, the serum level of total IgG, RF, an autoantibody, a cytokine/chemokine, an immune complex, or a marker of complement activation is measured 4 weeks, 16 weeks, or 24 weeks following administration of the FcRn antagonist.

In some embodiments, the subject shows a reduction in a serum level of an autoantibody following administration of the FcRn antagonist. In some embodiments, the autoantibody is an anti-Ro/SS-A antibody or an anti-La/SS-B antibody.

In some embodiments, the subject shows a reduction in a serum level of BAFF, type 1 interferon (IFN), IL 1B, IL 21, TNFα, IFNα, CD30, CD40 L, CCL5, CRP, and/or ferritin following administration of the FcRn antagonist, compared to a baseline value.

In some embodiments, the subject shows a reduction in a serum level of an immune complex following administration of the FcRn antagonist, compared to a baseline value. In some embodiments, the immune complex is a C1q immune complex.

In some embodiments, the subject shows a reduction in a serum level of a marker of complement activation. In some embodiments, the marker of complement activation is C3, C4, and/or split products thereof.

In some embodiments, the subject shows a change in saliva biomarker levels following administration of the FcRn antagonist, compared to a baseline value. In some embodiments, the saliva biomarker levels are measured 4 weeks, 16 weeks, or 24 weeks following administration of the FcRn antagonist.

In some embodiments, the subject shows a change in immunophenotype, focus score, number of germinal centers, lymphoepithelial lesions, and gene expression per mm² in parotid gland parenchyma following administration of the FcRn antagonist, compared to a baseline value. In some embodiments, the change in the immunophenotype, the focus score, the number of germinal centers, the lymphoepithelial lesions, and the gene expression per mm² is measured 24 weeks following administration of the FcRn antagonist.

In some embodiments, the subject shows a change in immunophenotyping in peripheral blood, optionally measured using flow cytometry, following administration of the FcRn antagonist, compared to a baseline value. In some embodiments, the immunophenotyping is measured at 4 weeks or 24 weeks following administration of the FcRn antagonist.

In some embodiments, the subject shows a change in gene expression profile in blood biomarkers, optionally measured using RNA sequencing, following administration of the FcRn antagonist, compared to a baseline value. In some embodiments, the gene expression profile is measured at 4 weeks, 16 weeks, or 24 weeks following administration of the FcRn antagonist.

The instant disclosure also provides an FcRn antagonist for use in the treatment of pSS, wherein the treatment is performed according to the methods described above and herein.

The instant disclosure also provides an FcRn antagonist for use in the manufacture of a medicament for the treatment of pSS, wherein the treatment is performed according to the methods described above and herein.

The instant disclosure also provides use of an FcRn antagonist for the treatment of pSS according to the methods described above and herein.

The instant disclosure also provides use of an FcRn antagonist for the manufacture of a medicament for treatment of pSS, wherein the treatment is performed according to the methods described above and herein.

### DETAILED DESCRIPTION

The present disclosure provides engineered FcRn antagonists and methods for their use in treating primary Sjögren's syndrome (pSS). Advantageously, the methods disclosed herein permit long-term reduction of pSS symptoms, reduction and/or prevention of glandular tissue destruction, and improvement in disease-related quality of life.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist” refers to any agent that binds specifically to FcRn and inhibits the binding of immunoglobulin to FcRn (e.g., human FcRn). In an embodiment, the FcRn antagonist is an Fc region (e.g., a variant Fc region disclosed herein) that specifically binds to FcRn through the Fc region and inhibits the binding of immunoglobulin to FcRn. In an embodiment, the FcRn antagonist is not a full-length IgG antibody. In an embodiment, the FcRn antagonist comprises an antigen binding site that binds a target antigen and a variant Fc region. In an embodiment, the FcRn antagonist is an Fc fragment comprising or consisting of an Fc region and lacking an antigen binding site. In an embodiment, the term “FcRn antagonist” refers to an antibody or antigen-binding fragment thereof that specifically binds to FcRn via its antigen binding domain or via its Fc region and inhibits the binding of the Fc region of immunoglobulin (e.g., IgG autoantibodies) to FcRn.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies (sdAb), monovalent antibodies, single chain antibodies or single-chain Fvs (scFv), camelid antibodies, affibody molecules, humanized antibodies, VHH fragments, Fab fragments, F(ab′) 2 fragments, disulfide-linked Fvs (sdFv), anti-idiotypic (anti-Id) antibodies (including, e.g., anti-anti-Id antibodies), and antigen-binding fragments of any of the above. Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂a or IgG_{2b}) of immunoglobulin molecule.

As used herein, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acid residues in length. In some embodiments, the hinge region is 15 amino acid residues in length. In some embodiments, the hinge region is 62 amino acid residues in length. Hinge regions can be subdivided into three distinct domains: upper, middle, and lower hinge domains. The FcRn antagonists of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 31).

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain constant region is an IgG1, IgG2, IgG3, or IgG4 heavy chain constant region. In some embodiments, the Fc region is formed from an IgG1 heavy chain constant region. In some embodiments, the IgG1 heavy chain constant region comprises a Glm1(a), Glm2(x), Glm3(f), or Glm17(z) allotype. See, e.g., Jefferis and Lefranc (2009) mAbs 1(4): 332-338, and de Taeye et al. (2020) Front Immunol. 11:740, incorporated herein by reference in their entirety.

As used herein, the term “variant Fc region” refers to an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein, the term “FcRn binding fragment” refers to a portion of an Fc region that is sufficient to confer FcRn binding.

As used herein, the term “EU position” refers to the amino acid position in the EU numbering convention for the Fc region described in Edelman, G M et al., Proc. Natl. Acad. USA, 63, 78-85(1969) and Rabat et al., in “Sequences of Proteins of Immunological Interest,” U.S. Dept. Health and Human Services, 5th edition, 1991.

As used herein, the term “baseline” refers to a measurement (e.g., IgG levels) in a patient (e.g., in a patient's blood or urine) prior to the first administration (e.g., intravenous or subcutaneous administration) of a treatment (e.g., an FcRn antagonist).

As used herein, the term “autoantibody-mediated disease” refers to any disease or disorder in which the underlying pathology is caused, at least in part, by pathogenic IgG autoantibodies.

As used herein, the terms “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the terms “dose” or “dosing” refer to an amount of an agent administered to a subject in a single administration.

As used herein, the terms “fixed dose” or “flat dose” both refer to a dose that does not vary based upon a characteristic (e.g., body mass, e.g., within a set range; sex; age, e.g., within a set range; etc.) of the subject.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of +5% of a given value or range, as are appropriate to perform the methods disclosed herein.

### Primary Sjögren's Syndrome

Primary Sjögren's syndrome (pSS) is a chronic, progressive autoimmune disease of unclear etiology, typically presented as an exocrinopathy. pSS is characterized by mononuclear inflammatory infiltrates and IgG plasma cells in salivary and lacrimal glands that lead to irreversible destruction of glandular tissue. Symptoms vary in type and intensity, but the burden of illness for many people with pSS is high. Although dry mouth and dry eyes are the most common symptoms, dryness can also occur in the nose, sinuses, ears, throat, skin, and, in women, the vagina. There is speculation that, in men with pSS, the prostate might be affected similarly to other organs. Patients may notice irritation, a gritty feeling, or painful burning in the eyes. Dry eyes are at increased risk for infection and susceptible to corneal damage if not treated. Dry mouth can lead to difficulty eating and swallowing dry foods, dental cavities, chipping, breaking, and loss of teeth. Dry mouth may also increase gingivitis and oral yeast infections that may cause pain and burning.

pSS can also affect the joints, muscles, nervous system (central nervous system and peripheral nervous system, including the autonomic nervous system), gastrointestinal tract (including the pancreas and liver), skin, blood vessels, lungs, and kidneys. Joint pain and stiffness with mild swelling are common, even in those without rheumatoid arthritis. Rashes may occur, including vasculitis, most commonly on the lower legs. Sun-sensitive rash is more common on the back, chest, face, and arms. Peripheral neuropathy can cause numbness and tingling, especially in the feet, and can frequently pre-date symptoms of dryness. Fatigue, cognitive dysfunction, and sleep abnormalities are frequently reported.

One of the most severe potential complications of pSS is lymphoma. Lymphoma occurs in up to 10% of pSS patients. The highest risk is for B-cell non-Hodgkin lymphoma, with the parotid gland being a common site. Other cancers occurring at a higher rate in pSS include multiple myeloma, thyroid, and stomach cancer.

Recognition of potential lung involvement in pSS has increased in recent years. Ten percent of patients are diagnosed with interstitial lung disease in the first year following diagnosis with pSS and 20% within five years. Cystic lung disease is identified more frequently in pSS compared to other connective tissue diseases. As many as 65% of pSS patients with no lung disease symptoms will have abnormal imaging studies.

Neurological manifestations may include numbness and tingling, especially in the feet and legs. However, the hands, face, and other areas may also be involved. Weakness and abnormal gait may occur in severe cases. Symptoms of dysautonomia in pSS patients can include heart rate abnormalities, sweating, blood pressure fluctuations, and difficulty with temperature regulation. Dysautonomia in pSS patients can also affect digestion, bladder control, and balance.

The cause of pSS is unknown but it is clearly an autoimmune disease. B cells play a central role in the immunopathogenesis and exhibit signs of hyperactivity. In addition, autoantibodies can create immune complexes that maintain and amplify the production of IFN alpha. This combination results in a cycle of immune activation that leads to tissue damage.

Diagnosis depends on a combination of symptoms, physical examination, blood tests, and special studies to look for objective evidence of dry eyes and dry mouth. Diagnosis of pSS cannot be based on symptoms alone because dry eyes and mouth are highly prevalent symptoms in the general population and can be caused by many other conditions or by medications. Instead, diagnosis can be based upon tests used to assess decreases in tear or saliva production. For example, staining of the cornea and/or conjunctiva with vital dyes can detect and assess for damage to the outer surface of the eyes caused by dryness. Presence of autoantibodies that are often associated with pSS can also be used in diagnosis. Typical autoantibodies include one or more of anti-nuclear antibodies (ANA), anti-Ro/SS-A antibodies, anti-La/SS-B antibodies, and rheumatoid factor (RF). Biopsy of the minor salivary glands may also be used to make a diagnosis when autoantibodies are absent.

Children and young adults often present with different signs and symptoms of pSS compared to older adults. Initial symptoms more commonly include swollen parotid glands and arthralgias. The pediatric population also may have neurologic and kidney manifestations. Dryness features might or might not be present at diagnosis.

Currently, no systemic immunomodulating therapies are approved for pSS. Hydroxychloroquine may be helpful in some pSS patients to reduce joint pain, fatigue, or rash. Patients with systemic problems, such as fever, severe rash, lung disease, neurologic problems, or kidney involvement may be treated with corticosteroids (such as prednisone and methylprednisolone) and/or immunosuppressive drugs like methotrexate, azathioprine, mycophenolate mofetil, leflunomide, or cyclophosphamide. In addition, biological therapies, such as rituximab, may be used, especially in severe disease.

Dry eyes usually respond to artificial tears applied regularly during the day or to ointment applied at night. Other measures, such as plugging or blocking tear ducts by the ophthalmologist, can be used in more severe cases. Eye drops that reduce inflammation in the glands around the eyes, such as cyclosporine and lifitegrast, may significantly improve symptoms and decrease the need for artificial tears. Many patients benefit from using prescription medications that stimulate saliva flow, such as pilocarpine or cevimeline. If patients develop yeast infections, anti-fungal therapies may be needed. Medications that reduce gastric acid (such as proton-pump inhibitors and H2 blockers) may lessen symptoms of acid reflux.

There is a clear need for more effective treatments, given that the currently used medications treat only the symptoms of pSS and all have side effects which prohibit their continued use.

### FcRn Antagonists

FcRn antagonists that are useful in the methods and uses provided herein include any molecule that binds to and inhibits FcRn, including, but not limited to, any anti-FcRn antibody, any anti-FcRn binding region, or any Fc domain or Fc region.

In some embodiments, the FcRn antagonists disclosed herein comprise two, three, or four FcRn binding regions, such as an Fc region.

Any Fc region can be altered to produce a variant Fc region for use in the methods disclosed herein. In general, an Fc region, or FcRn binding fragment thereof, is from a human immunoglobulin. It is understood, however, that the Fc region may be derived from an immunoglobulin of any other mammalian species, including for example, a camelid species, a rodent (e.g., a mouse, rat, rabbit, guinea pig), or non-human primate (e.g., chimpanzee, macaque) species. Moreover, the Fc region or portion thereof may be derived from any immunoglobulin class, including IgM, IgG, IgD, IgA, and IgE, and any immunoglobulin isotype, including IgG1, IgG2, IgG3, and IgG4. In an embodiment, the Fc region is an IgG Fc region (e.g., a human IgG region). In an embodiment, the Fc region is an IgG1 Fc region (e.g., a human IgG1 region). In an embodiment, the Fc region is a chimeric Fc region comprising portions of several different Fc regions. Suitable examples of chimeric Fc regions are set forth in US 2011/0243966A1, which is incorporated herein by reference in its entirety. A variety of Fc region gene sequences (e.g., human constant region gene sequences) are available in the form of publicly accessible deposits.

An Fc region can be further truncated or internally deleted to produce a minimal FcRn binding fragment thereof. The ability of an Fc-region fragment to bind to FcRn can be determined using any art recognized binding assay (e.g., ELISA).

To enhance the manufacturability of the FcRn antagonists disclosed herein, it is preferable that the constituent Fc regions do not comprise any non-disulfide bonded cysteine residues. Accordingly, in an embodiment, the Fc regions do not comprise a free cysteine residue.

Any Fc variant, or FcRn binding fragment thereof, that binds specifically to FcRn with increased affinity and reduced pH dependence relative to the native (i.e., wild-type) Fc region can be used in the methods disclosed herein. In an embodiment, the variant Fc region comprises amino acid alterations, substitutions, insertions, and/or deletions that confer the desired characteristics. In some embodiments, the FcRn antagonist comprises a variant Fc region, or FcRn binding fragment thereof, which binds to FcRn with a higher affinity at pH 5.5 as compared to a corresponding wild-type Fc region. In some embodiments, the FcRn antagonist comprises or consists of a variant Fc region, or FcRn binding fragment thereof, which binds to FcRn with a higher affinity at pH 6.0 and/or at pH 7.4 as compared to a corresponding wild-type Fc region. In some embodiments, the FcRn antagonist comprises a variant Fc region, or FcRn binding fragment thereof, which binds to FcRn with a higher affinity at both acidic and neutral pH.

In some embodiments, the variant Fc region is derived from the Fc region of any native immunoglobulin. In some embodiments, the native immunoglobulin is a human immunoglobulin. In some embodiments, the immunoglobulin is IgA, IgD, IgE, or IgG. In some embodiments, the immunoglobulin is IgG. In some embodiments, the immunoglobulin is human IgA, human IgD, human IgE, or human IgG. In some embodiments, the immunoglobulin is human IgG. In some embodiments, the IgG is IgG1, IgG2, IgG3, or IgG4. In some embodiments, the human IgG is human IgG1, human IgG2, human IgG3, or human IgG4. In some embodiments, the variant Fc region varies from the human IgG1 Fc region. In some embodiments, the human IgG1 Fc region comprises a Glm1(a), Glm2(x), Glm3(f), or Glm17(z) allotype.

In an embodiment, the variant Fc region, or FcRn binding fragment thereof consists of two Fc domains.

In an embodiment, the variant Fc region comprises or consists of a first Fc domain and a second Fc domain which form a homodimer or heterodimer. In an embodiment, the first Fc domain and/or the second Fc domain comprise amino acids Y, T, E, K, and F at EU positions 252, 254, 256, 433, and 434, respectively. In an embodiment, the first Fc domain and/or the second Fc domain comprise amino acids Y, T, E, K, F, and Y at EU positions 252, 254, 256, 433, 434, and 436, respectively.

In some embodiments, the FcRn antagonists disclosed herein comprise or consist of at least one Fc domain, wherein the amino acid sequence of the at least one Fc domain comprises or consists of the amino acid sequence of SEQ ID NO: 1, provided below.

TABLE 1

| | SEQ |
|---|---|
| Amino Acid Sequence | ID NO: |
| X₁X₂X₃X₄X₅X₆PPCPAPELLGGPSVFLFPPKPKDTLYITR | 1 |
| EPEVTCVVVDVSHEDPEVKFNWYVDGVEVHNAKTKPREEQY | |
| NSTYRVVSVLTVLHQDWLNGKEYKCKVSNKALPAPIEKTIS | |
| KAKGQPREPQVYTLPPSRDELTKNQVSLTCLVKGFYPSDIA | |
| VEWESNGQPENNYKTTPPVLDSDGSFFLYSKLTVDKSRWQQ | |
| GNVFSCSVMHEALKFHYTQKSLSLSPX₇X8 | |

wherein:
X₁ is D or absent;
X₂ is K or absent;
X₃ is T or absent;
X₄ is H or absent;
X₅ is T or absent;
X₆ is C or absent;
X₇ is G or absent;
X₈ is K or absent.

In some embodiments, the FcRn antagonists disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 1.

In some embodiments, the FcRn antagonists disclosed herein comprise or consist of an amino acid sequence at least 70%, 75%, 80%, 85%, 90%, 95%, 96%, 97%, 98%, 99%, or 100% identical to the amino acid sequence of a variant Fc region comprising or consisting of a dimer of a first Fc domain and a second Fc domain, wherein the amino acid sequence of the first and second Fc domain comprises or consists of an amino acid sequence independently selected from the group consisting of SEQ ID NOs: 2-22. In some embodiments, the dimer is a heterodimer or a homodimer.

TABLE 2

| Amino Acid Sequence | SEQ ID NO: |
|---|---|
| DKTHTCPPCPAPELLGGPSVFLFPPKPKDTLYITREPEVTCVVVDVSHEDP | 2 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDP | 3 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| DKTHTCPPCPAPELLGGPSVFLFPPKPKDTLYITREPEVTCVVVDVSHEDP | 4 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPE | 5 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSPGK | |
| KTHTCPPCPAPELLGGPSVFLFPPKPKDTLYITREPEVTCVVVDVSHEDPE | 6 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSPG | |
| KTHTCPPCPAPELLGGPSVFLFPPKPKDTLYITREPEVTCVVVDVSHEDPE | 7 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSP | |
| THTCPPCPAPELLGGPSVFLFPPKPKDTLYITREPEVTCVVVDVSHEDPEV | 8 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| THTCPPCPAPELLGGPSVFLFPPKPKDTLYITREPEVTCVVVDVSHEDPEV | 9 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| THTCPPCPAPELLGGPSVFLFPPKPKDTLYITREPEVTCVVVDVSHEDPEV | 10 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| TCPPCPAPELLGGPSVFLFPPKPKDTLYITREPEVTCVVVDVSHEDPEVKF | 11 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKF | 12 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKE | 13 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNW | 14 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNW | 15 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNW | 16 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVK | 17 |
| FNWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSPGK | |
| HTCPPCPAPELLGGPSVFLFPPKPKDTLYITREPEVTCVVVDVSHEDPEVK | 18 |
| ENWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSPG | |
| HTCPPCPAPELLGGPSVFLFPPKPKDTLYITREPEVTCVVVDVSHEDPEVK | 19 |
| FNWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSP | |
| CPPCPAPELLGGPSVFLFPPKPKDTLYITREPEVTCVVVDVSHEDPEVKFN | 20 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSPG | |
| CPPCPAPELLGGPSVFLFPPKPKDTLYITREPEVTCVVVDVSHEDPEVKFN | 21 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSPGK | |
| CPPCPAPELLGGPSVFLFPPKPKDTLYITREPEVTCVVVDVSHEDPEVKFN | 22 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSP | |

In an embodiment, the first Fc domain and/or the second Fc domain comprise an amino acid sequence independently selected from the group consisting of SEQ ID NOs: 2, 3, 20, or 21. In an embodiment, the first Fc domain and the second Fc domain comprise an amino acid sequence independently selected from the group consisting of SEQ ID NOs: 2, 3, 20, or 21. In some embodiments, the FcRn antagonist comprises a population of FcRn antagonist molecules. In some embodiments, a FcRn antagonist comprising a first Fc domain and a second Fc domain comprising an amino acid sequence independently selected from the group consisting of SEQ ID NOs: 2, 3, 20, or 21 is the predominant FcRn antagonist molecule in the population of FcRn antagonist molecules. In some embodiments, the predominant FcRn antagonist molecule makes up at least 50%, 55%, 60%, 65%, 70%, 75%, 80%, 85%, 90%, 95%, 96%, 97%, 98%, or 99% of the population of FcRn antagonist molecules.

In an embodiment, the amino acid sequence of the Fc domains of the variant Fc region comprises the amino acid sequence of SEQ ID NO: 2. In an embodiment, the amino acid sequence of the Fc domains of the variant Fc region consists of the amino acid sequence of SEQ ID NO: 2. In an embodiment, the amino acid sequence of the Fc domains of the variant Fc region comprises the amino acid sequence of SEQ ID NO: 3. In an embodiment, the amino acid sequence of the Fc domains of the variant Fc region consists of the amino acid sequence of SEQ ID NO: 3. In an embodiment, the amino acid sequence of the Fc domains of the variant Fc region comprises the amino acid sequence of SEQ ID NO: 20. In an embodiment, the amino acid sequence of the Fc domains of the variant Fc region consists of the amino acid sequence of SEQ ID NO: 20. In an embodiment, the amino acid sequence of the Fc domains of the variant Fc region comprises the amino acid sequence of SEQ ID NO: 21. In an embodiment, the amino acid sequence of the Fc domains of the variant Fc region consists of the amino acid sequence of SEQ ID NO: 21.

In certain embodiments, the variant Fc region is a heterodimer, where the constituent Fc domains are different from each other. Methods of producing Fc heterodimers are known in the art (see, e.g., U.S. Pat. No. 8,216,805, which is incorporated by reference herein in its entirety). In an embodiment, the FcRn antagonist consists of a variant Fc region, wherein the variant Fc region consists of two Fc domains which form a heterodimer, wherein the amino acid sequence of each of the Fc domains is independently selected from SEQ ID NOs: 2, 3, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 2 and the amino acid sequence of the second Fc domain consists of or comprises the amino acid sequence of SEQ ID NOs: 3, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 3 and the amino acid sequence of the second Fc domain consists of or comprises the amino acid sequence of SEQ ID NOs: 2, 20, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 20 and the amino acid sequence of the second Fc domain consists of or comprises the amino acid sequence of SEQ ID NOs: 2, 3, or 21. In an embodiment, the FcRn antagonist consists of or comprises a variant Fc region, wherein the variant Fc region consists of or comprises two Fc domains which form a heterodimer, wherein the amino acid sequence of the first Fc domain consists of or comprises the amino acid sequence of SEQ ID NO: 21 and the amino acid sequence of the second Fc domain consists of or comprises the amino acid sequence of SEQ ID NOs: 2, 3, or 20.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 2.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 3.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 20.

In an embodiment, the FcRn antagonist consists of or comprises a variant Fc region, wherein the variant Fc region consists of or comprises two Fc domains which form a homodimer, wherein the amino acid sequence of each of the Fc domains consists of or comprises the amino acid sequence of SEQ ID NO: 21.

In some embodiments, the FcRn antagonist comprises glycanation on one or both of the Fc domains. In some embodiments, the FcRn antagonist molecules comprise glycanation at EU position 297 on one or both of the Fc domains. In some embodiments, the glycanation comprises an N-glycan. In some embodiments, the N-glycan comprises a GOF N-glycan, GIF N-glycan, G2F N-glycan, or GO N-glycan.

In some embodiments, FcRn antagonist comprises or consists of a population of FcRn antagonists, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonists comprise galactose. In some embodiments, the population comprises or consists of FcRn antagonists, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonists comprise fucose.

In some embodiments, the FcRn antagonist lacks an amino acid at EU position 441 of one or both Fc domains. In some embodiments, the FcRn antagonist comprises glycine and lysine at EU positions 440 and 441, respectively. In some embodiments, the FcRn antagonist lacks amino acids at EU positions 440 and 441. In some embodiments, the FcRn antagonist comprises amidated proline at EU position 439. In some embodiments, the FcRn antagonist lacks amino acids at EU positions 440 and 441 and comprise amidated proline at EU position 439.

In some embodiments, the FcRn antagonist comprises aspartate, lysine, threonine, histidine, threonine, and cysteine at EU positions 221, 222, 223, 224, 225, and 226, respectively. In some embodiments, the FcRn antagonist lacks an amino acid at EU positions 221, and comprises lysine, threonine, histidine, threonine, and cysteine at EU positions 222, 223, 224, 225, and 226, respectively. In some embodiments, the FcRn antagonist lacks amino acids at EU positions 221 and 222, and comprises threonine, histidine, threonine, and cysteine at EU positions 223, 224, 225, and 226, respectively. In some embodiments, the FcRn antagonist lacks amino acids at EU positions 221-224, and comprises threonine and cysteine at EU positions 225 and 226, respectively. In some embodiments, the FcRn antagonist lacks amino acids at EU positions 221, 222, 223, 224, 225, and 226.

In some embodiments, the FcRn antagonist is a population of FcRn antagonist molecules. In some embodiments, the population of FcRn antagonist molecules comprises one or more of the FcRn antagonists described herein. In some embodiments, the FcRn antagonist is any of those described in U.S. Patent Application No. 63/383,599, filed on Nov. 14, 2022, incorporated herein by reference in its entirety. In some embodiments, the FcRn antagonist is a population of FcRn antagonists as described in U.S. Patent Application No. 63/383,599, filed on Nov. 14, 2022, incorporated herein by reference in its entirety.

In an embodiment, the FcRn antagonist is efgartigimod (CAS Registry No. 1821402-21-4). The term “efgartigimod” as used herein is interchangeable with “efgartigimod alfa.” In some embodiments, efgartigimod is efgartigimod alfa-fcab.

In an embodiment, the anti-FcRn antibody is nipocalimab (M281), rozanolixizumab (UCB7665), orilanolimab (ALXN1830/SYNT001), or batoclimab (IMVT-1401/RVT1401/HBM9161).

In an embodiment, an antibody that binds specifically to FcRn and inhibits the binding of the Fc region of immunoglobulin to FcRn is nipocalimab, also known as M281. Nipocalimab is a full-length “Fc dead” IgG1 monoclonal antibody. Nipocalimab has been administered as an intravenous infusion in Phase 2 clinical trials for the treatment of myasthenia gravis (MG), warm autoimmune hemolytic anemia (WAIHA), and hemolytic disease of fetus and newborn (HDFN). Nipocalimab comprises the light chain (SEQ ID NO: 23) and heavy chain (SEQ ID NO: 24) sequences set forth in Table 3 below (VL region of SEQ ID NO: 23 and VH region of SEQ ID NO: 24 are underlined):

TABLE 3
Heavy chain and light chain sequences of nipocalimab

| SEQ ID NO: | Amino Acid Sequence |
|---|---|
| 23 | QSALTQPASVSGSPGQSITISCTGTGSDVGSYNLVSWYQQHPGKAP |
| (light chain | KLMIYGDSERPSGVSNRFSGSKSGNTASLTISGLQAEDEADYYCSS |
| sequence; | YAGSGIYVFGTGTKVTVLGQPKAAPSVTLFPPSSEELQANKATLVC |
| VL region | LISDFYPGAVTVAWKADSSPVKAGVETTTPSKQSNNKYAASSYLSL |
| underlined) | TPEQWKSHKSYSCQVTHEGSTVEKTVAPTECS |
| 24 | EVQLLESGGGLVQPGGSLRLSCAASGFTFSTYAMGWVRQAPGKGLE |
| (heavy chain | WVSSIGASGSQTRYADSVKGRFTISRDNSKNTLYLQMNSLRAEDTA |
| sequence; | VYYCARLAIGDSYWGQGTMVTVSSASTKGPSVFPLAPSSKSTSGGT |
| VH region | AALGCLVKDYFPEPVTVSWNSGALTSGVHTFPAVLQSSGLYSLSSV |
| underlined) | VTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNW |
| | YVDGVEVHNAKTKPREEQYASTYRVVSVLTVLHQDWLNGKEYKCKV |
| | SNKALPAPIEKTISKAKGQPREPQVYTLPPSREEMTKNQVSLTCLV |
| | KGFYPSDIAVEWESNGQPENNYKTTPPVLDSDGSFFLYSKLTVDKS |
| | RWQQGNVFSCSVMHEALHNHYTQKSLSLSPG |

In an embodiment, an antibody that binds specifically to FcRn and inhibits the binding of the Fc region of immunoglobulin to FcRn is rozanolixizumab, also known as UCB 7665. Rozanolixizumab is a full-length humanized IgG4 monoclonal antibody. Rozanolixizumab has been administered as a subcutaneous infusion in ongoing clinical trials for MG, immune thrombocytopenia (ITP), and chronic inflammatory demyelinating polyneuropathy (CIDP). Rozanolixizumab comprises the light chain (SEQ ID NO: 25) and heavy chain (SEQ ID NO: 26) sequences set forth in Table 4 below (VL region of SEQ ID NO: 25 and VH region of SEQ ID NO: 26 are underlined):

TABLE 4
Heavy chain and light chain sequences of rozanolixizumab

| SEQ ID NO: | Amino Acid Sequence |
|---|---|
| 25 | DIQMTQSPSSLSASVGDRVTITCKSSQSLVGASGKTYLYWLFQKP |
| (light chain | GKAPKRLIYLVSTLDSGIPSRFSGSGSGTEFTLTISSLQPEDFAT |
| sequence; | YYCLQGTHFPHTFGQGTKLEIKRTVAAPSVFIFPPSDEQLKSGTA |
| VL region | SVVCLLNNFYPREAKVQWKVDNALQSGNSQESVTEQDSKDSTYSL |
| underlined) | SSTLTLSKADYEKHKVYACEVTHQGLSSPVTKSFNRGEC |
| 26 | EVPLVESGGGLVQPGGSLRLSCAVSGFTFSNYGMVWVRQAPGKGL |
| (heavy chain | EWVAYIDSDGDNTYYRDSVKGRFTISRDNAKSSLYLQMNSLRAED |
| sequence; | TAVYYCTTGIVRPFLYWGQGTLVTVSSASTKGPSVFPLAPCSRST |
| VH region | SESTAALGCLVKDYFPEPVTVSWNSGALTSGVHTFPAVLQSSGLY |
| underlined) | SLSSVVTVPSSSLGTKTYTCNVDHKPSNTKVDKRVESKYGPPCPP |
| | CPAPEFLGGPSVFLFPPKPKDTLMISRTPEVTCVVVDVSQEDPEV |
| | QFNWYVDGVEVHNAKTKPREEQFNSTYRVVSVLTVLHQDWLNGKE |
| | YKCKVSNKGLPSSIEKTISKAKGQPREPQVYTLPPSQEEMTKNQV |
| | SLTCLVKGFYPSDIAVEWESNGQPENNYKTTPPVLDSDGSFFLYS |
| | RLTVDKSRWQEGNVFSCSVMHEALHNHYTQKSLSLSLGK |

In an embodiment, an antibody that binds specifically to FcRn and inhibits the binding of the Fc region of immunoglobulin to FcRn is orilanolimab, also known as SYNT001. Orilanolimab is another full-length humanized IgG4 monoclonal antibody. Orilanolimab has been administered as an intravenous infusion in Phase 2 clinical trials for treatment of WAIHA. Orilanolimab comprises the light chain (SEQ ID NO: 27) and heavy chain (SEQ ID NO: 28) sequences set forth in Table 5 below (VL region of SEQ ID NO: 27 and VH region of SEQ ID NO: 28 are underlined):

TABLE 5
Heavy chain and light chain sequences of orilanolimab

| SEQ ID NO: | Amino Acid Sequence |
|---|---|
| 27 | DIQMTQSPSSLSASVGDRVTITCKASDHINNWLAWYQQKPGQAPR |
| (light chain | LLISGATSLETGVPSRFSGSGTGKDYTLTISSLQPEDFATYYCQQ |
| sequence; | YWSTPYTFGGGTKVEIKRTVAAPSVFIFPPSDEQLKSGTASVVCL |
| VL region | LNNFYPREAKVQWKVDNALQSGNSQESVTEQDSKDSTYSLSSTLT |
| underlined) | LSKADYEKHKVYACEVTHQGLSSPVTKSFNRGEC |
| 28 | QVQLVQSGAELKKPGASVKLSCKASGYTFTSYGISWVKQATGQGL |
| (heavy chain | EWIGEIYPRSGNTYYNEKFKGRATLTADKSTSTAYMELRSLRSED |
| sequence; | SAVYFCARSTTVRPPGIWGTGTTVTVSSASTKGPSVFPLAPCSRS |
| VH region | TSESTAALGCLVKDYFPEPVTVSWNSGALTSGVHTFPAVLQSSGL |
| underlined) | YSLSSVVTVPSSSLGTKTYTCNVDHKPSNTKVDKRVESKYGPPCP |
| | PCPAPEFLGGPSVFLFPPKPKDTLMISRTPEVTCVVVDVSQEDPE |
| | VQFNWYVDGVEVHNAKTKPREEQFNSTYRVVSVLTVLHQDWLNGK |
| | EYKCKVSNKGLPSSIEKTISKAKGQPREPQVYTLPPSQEEMTKNQ |
| | VSLTCLVKGFYPSDIAVEWESNGQPENNYKTTPPVLDSDGSFFLY |
| | SRLTVDKSRWQEGNVFSCSVMHEALHNHYTQKSLSLSLG |

In an embodiment, an antibody that binds specifically to FcRn and inhibits the binding of the Fc region of immunoglobulin to FcRn is batoclimab, also known as IMVT1401/RVT1401/HBM9161. Batoclimab is another full-length “Fc dead” IgG1 monoclonal antibody. Batoclimab has been administered as a subcutaneous injection in ongoing Phase 2 clinical trials for treatment of MG and Graves' ophthalmopathy. Batoclimab comprises the light chain (SEQ ID NO: 29) and heavy chain (SEQ ID NO: 30) sequences set forth in Table 6 below (VL region of SEQ ID NO: 29 and VH region of SEQ ID NO: 30 are underlined):

TABLE 6
Heavy chain and light chain sequences of batoclimab

| SEQ ID NO: | Amino Acid Sequence |
|---|---|
| 29 | SYVLTQSPSVSVAPGQTARITCGGNNIGSKSVHWYQQKPGQAPVL |
| (light chain | VVYDDSDRPSGIPERFSASNSGNTATLTISRVEAGDEADYYCQVW |
| sequence; | DSSSDHVVFGGGTKLTVLGQPKAAPSVTLFPPSSEELQANKATLV |
| VL region | CLISDFYPGAVTVAWKADSSPVKAGVETTTPSKQSNNKYAASSYL |
| underlined) | SLTPEQWKSHRSYSCQVTHEGSTVEKTVAPTECS |
| 30 | QLLLQESGPGLVKPSETLSLTCTVSGGSLSSSFSYWVWIRQPPGK |
| (heavy chain | GLEWIGTIYYSGNTYYNPSLKSRLTISVDTSKNHFSLKLSSVTAA |
| sequence; | DTAVYYCARRAGILTGYLDSWGQGTLVTVSSASTKGPSVFPLAPS |
| VH region | SKSTSGGTAALGCLVKDYFPEPVTVSWNSGALTSGVHTFPAVLQS |
| underlined) | SGLYSLSSVVTVPSSSLGTQTYICNVNHKPSNTKVDKRVEPKSCD |
| | KTHTCPPCPAPEAAGGPSVFLFPPKPKDTLMISRTPEVTCVVVDV |
| | SHEDPEVKFNWYVDGVEVHNAKTKPREEQYNSTYRVVSVLTVLHQ |
| | DWLNGKEYKCKVSNKALPAPIEKTISKAKGQPREPQVYTLPPSRE |
| | EMTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |

### Pharmaceutical Compositions

In an aspect, the instant disclosure provides pharmaceutical compositions comprising an FcRn antagonist for use in methods of treating pSS. In certain embodiments, these compositions comprise or consist of a variant Fc region, or FcRn binding fragment thereof, that binds specifically to FcRn, particularly human FcRn, with increased affinity and reduced pH dependence relative to a native Fc region. In other embodiments, the FcRn antagonist composition is an antibody or antigen-binding fragment thereof that binds specifically to FcRn via its antigen binding domain and inhibits the binding of Fc region of immunoglobulin to FcRn. In general, these FcRn antagonists inhibit the binding of Fc-containing agents (e.g., antibodies and immunoadhesins) to FcRn in vivo, which results in an increased rate of degradation of the Fc-containing agents and, concomitantly, a reduced serum level of these agents.

In an embodiment, the FcRn antagonist is efgartigimod. Efgartigimod (ARGX-113) is a modified human immunoglobulin (Ig) gamma (IgG) 1-derived Fc of the za allotype that binds with nanomolar affinity to human FcRn. Efgartigimod encompasses the IgG1 Fc-region and has been engineered using ABDEG™ technology to increase its affinity for FcRn at both physiological and acidic pH. The increased affinity for FcRn of efgartigimod at both acidic and physiological pH results in a blockage of FcRn-mediated recycling of IgG.

Due to its increased affinity for FcRn at both acidic and neutral pH, efgartigimod blocks the FcRn/IgG complex from forming, which results in degradation of endogenous IgGs, including autoantibodies that cause IgG-mediated autoimmune diseases. This blocking of FcRn by efgartigimod results in a rapid and profound reduction in autoantibody levels, which underlies the therapeutic strategy for the treatment of autoimmune indications where IgG autoantibodies are expected to have a central role in the disease pathology.

Efgartigimod is under development for both the intravenous (IV) and subcutaneous (SC) administration route.

For IV administration, in certain embodiments, efgartigimod may be administered in a formulation comprising sodium phosphate, sodium chloride, L-arginine hydrochloride, and polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80. In certain embodiments, efgartigimod may be administered in a formulation comprising about 25 mM sodium phosphate, about 100 mM sodium chloride, and about 150 mM L-arginine hydrochloride (pH 6.7), with about 0.02% (w/v) polysorbate 80, via intravenous infusion in a total volume of about 250 mL over a period of about 2 hours. In certain embodiments, efgartigimod may be administered in a formulation comprising 25 mM sodium phosphate, 100 mM sodium chloride, and 150 mM L-arginine hydrochloride (pH 6.7), with 0.02% (w/v) polysorbate 80, via intravenous infusion in a total volume of 250 mL over a period of 2 hours. See, e.g., WO2019110823A1, which is incorporated by reference herein in its entirety.

In certain embodiments, efgartigimod may be administered in a formulation comprising an aqueous solution comprising about 25 mM sodium phosphate, about 100 mM sodium chloride, and about 150 mM L-arginine hydrochloride with a pH of about 6.7, with about 0.02% (w/v) polysorbate 80, diluted for intravenous infusion to a total volume of about 125 mL over a period of about 1 hour. In certain embodiments, efgartigimod may be administered in a formulation comprising an aqueous solution comprising 25 mM sodium phosphate, 100 mM sodium chloride, and 150 mM L-arginine hydrochloride with a pH of 6.7, with 0.02% (w/v) polysorbate 80, diluted for intravenous infusion to a total volume of 125 mL over a period of 1 hour.

In certain embodiments, efgartigimod may be administered in a formulation comprising an aqueous solution comprising about 4 mM sodium phosphate, about 146 mM sodium chloride, about 24 mM L-arginine, and about 0.0032% (w/v) polysorbate 80, with a pH of about 6.7. This formulation is administered via intravenous infusion in a total volume of about 125 mL over a period of about 1 hour. In certain embodiments, efgartigimod may be administered in a formulation comprising an aqueous solution comprising 4 mM sodium phosphate, 146 mM sodium chloride, 24 mM L-arginine, and 0.0032% (w/v) polysorbate 80, with a pH of 6.7. This formulation is administered via intravenous infusion in a total volume of 125 mL over a period of 1 hour.

In certain embodiments, efgartigimod is administered via IV infusion and is provided in a sterile, colorless, clear concentrate solution at a concentration of about 20 mg/mL. In certain embodiments, efgartigimod is administered via IV infusion and is provided in a sterile, colorless, clear concentrate solution at a concentration of 20 mg/mL.

In certain embodiments, efgartigimod is administered via IV infusion and is provided in a vial (e.g., a single-dose vial). In certain embodiments, a vial of efgartigimod contains about 400 mg of efgartigimod at a concentration of about 20 mg/mL. In certain embodiments, a vial of efgartigimod contains 400 mg of efgartigimod at a concentration of 20 mg/mL. In certain embodiments, each mL of solution in a vial of efgartigimod contains about 31.6 mg L-arginine hydrochloride, about 0.2 mg polysorbate 80, about 5.8 mg sodium chloride, about 2.4 mg sodium phosphate dibasic anhydrous, about 1.1 mg sodium phosphate monobasic monohydrate, and water for injection, USP, at a pH of about 6.7. In certain embodiments, each mL of solution in a vial of efgartigimod contains 31.6 mg L-arginine hydrochloride, 0.2 mg polysorbate 80, 5.8 mg sodium chloride, 2.4 mg sodium phosphate dibasic anhydrous, 1.1 mg sodium phosphate monobasic monohydrate, and water for injection, USP, at a pH of 6.7.

In certain embodiments, for patients weighing under 120 kg, efgartigimod is administered at a dose of about 10 mg/kg as an IV infusion. In certain embodiments, for patients weighing under 120 kg, efgartigimod is administered at a dose of about 10 mg/kg as an IV infusion over about one hour. In certain embodiments, for patients weighing under 120 kg, efgartigimod is administered at a dose of about 10 mg/kg as an IV infusion over about one hour once weekly. In certain embodiments, for patients weighing under 120 kg, efgartigimod is administered at a dose of about 10 mg/kg as an IV infusion over about one hour once weekly for about 4 weeks. In certain embodiments, for patients weighing under 120 kg, efgartigimod is administered at a dose of 10 mg/kg as an IV infusion. In certain embodiments, for patients weighing under 120 kg, efgartigimod is administered at a dose of 10 mg/kg as an IV infusion over one hour. In certain embodiments, for patients weighing under 120 kg, efgartigimod is administered at a dose of 10 mg/kg as an IV infusion over one hour once weekly. In certain embodiments, for patients weighing under 120 kg, efgartigimod is administered at a dose of 10 mg/kg as an IV infusion over one hour once weekly for 4 weeks. In certain embodiments, for patients weighing 120 kg or more, efgartigimod is administered at a dose of about 1200 mg per IV infusion. In certain embodiments, for patients weighing 120 kg or more, efgartigimod is administered at a dose of 1200 mg per IV infusion.

For SC administration, in certain embodiments, efgartigimod may be administered alone. Alternatively, for SC administration, in certain embodiments, efgartigimod may be administered co-formulated with hyaluronidase, for example, rHuPH20. The co-formulated material will allow SC dosing of larger volumes.

In some embodiments, efgartigimod may be administered in a formulation comprising an aqueous solution comprising about 20 mM L-histidine, about 100 mM sodium chloride, about 60 mM sucrose, about 10 mM L-methionine, and about 0.04% (w/v) polysorbate 20, wherein the formulation has a pH of about 6.0. In some embodiments, the formulation comprises about 180 mg/mL efgartigimod. In some embodiments, efgartigimod may be administered in a formulation comprising an aqueous solution comprising 20 mM L-histidine, 100 mM sodium chloride, 60 mM sucrose, 10 mM L-methionine, and 0.04% (w/v) polysorbate 20, wherein the formulation has a pH of 6.0. In some embodiments, the formulation comprises 180 mg/mL efgartigimod.

In some embodiments, efgartigimod may be administered in a formulation comprising an aqueous solution comprising about 20 mM L-histidine, about 50 mM L-arginine, about 100 mM sodium chloride, about 60 mM sucrose, about 10 mM L-methionine, and about 0.04(w/v) polysorbate 80, wherein the formulation has a pH of about 6.0. In some embodiments, the formulation comprises about 200 mg/mL efgartigimod. In some embodiments, efgartigimod may be administered in a formulation comprising an aqueous solution comprising 20 mM L-histidine, 50 mM L-arginine, 100 mM sodium chloride, 60 mM sucrose, 10 mM L-methionine, and 0.04(w/v) polysorbate 80, wherein the formulation has a pH of 6.0. In some embodiments, the formulation comprises 200 mg/mL efgartigimod.

rHuPH20 is the active ingredient of Halozyme's commercial product HYLENEX® recombinant (hyaluronidase human injection), referred to as HYLENEX®, which was approved by FDA for marketed use in the U.S. in December 2005. HYLENEX® is a tissue permeability modifier indicated as an adjuvant in SC fluid administration for achieving hydration, to increase the dispersion and absorption of other injected drugs, and in SC urography, for improving resorption of radiopaque agents.

rHuPH20 is a recombinant enzyme human hyaluronidase produced by genetically engineered Chinese hamster ovary (CHO) cells containing a deoxyribonucleic plasmid encoding a soluble fragment of human hyaluronidase (posterior head protein 20 [PH20]).

The HZ202 rHuPH20 DS is currently registered in HYLENEX® and other biologic drug products co-formulated with rHuPH20 DS. As such, in certain embodiments HZ202 rHuPH20 DS is used in the efgartigimod/rHuPH20 co-formulated product for SC administration (i.e., efgartigimod PH20 SC).

Provided in the co-formulations, combinations, uses and methods herein are soluble hyaluronidases. Soluble hyaluronidases include any that, upon expression, are secreted from a cell and exist in soluble form. Such soluble hyaluronidases include, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

rHuPH20 refers to the composition produced upon expression in a cell, such as a CHO cell, of nucleic acid encoding residues 36-482 of SEQ ID NO:32, generally linked to the native or a heterologous signal sequence (residues 1-35 of SEQ ID NO:32). rHuPH20 is produced by expression of a nucleic acid molecule, such as encoding amino acids 1-482(set forth in SEQ ID NO: 32) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO:32, and some shorter polypeptides, in various abundance. Typically, rHuPH20 is produced in cells that facilitate correct N-glycosylation to retain activity, such as CHO cells (e.g., DG44 CHO cells). In some embodiments, one of the most abundant species is the 446 amino acid polypeptide corresponding to residues 36-481 of SEQ ID NO:32. Also included are polypeptides that are soluble or secreted upon expression in a mammalian cell and have at least 90%, 91%, 92%, 93%, 94%, 95%, 96%, 97%, 98%, 99%, or more sequence identity with residues 36-482 of SEQ ID NO:32.

In some embodiments, the pharmaceutical formulation comprises an FcRn antagonist in an amount from about 20 mg to about 20,000 mg. In some embodiments, the pharmaceutical formulation comprises an FcRn antagonist in an amount from about 200 mg to about 20,000 mg. In some embodiments, the pharmaceutical formulation comprises an FcRn antagonist in an amount from about 300 mg to about 6000 mg. In some embodiments, the pharmaceutical formulation comprises an FcRn antagonist in an amount from about 750 mg to about 3000 mg. In some embodiments, the pharmaceutical formulation comprises an FcRn antagonist in an amount from about 1000 mg to about 2500 mg. In some embodiments, the pharmaceutical formulation comprises an FcRn antagonist in an amount from about 1000 mg to about 2000 mg.

In some embodiments, the pharmaceutical formulation comprises an FcRn antagonist in an amount from 20 mg to 20,000 mg. In some embodiments, the pharmaceutical formulation comprises an FcRn antagonist in an amount from 200 mg to 20,000 mg. In some embodiments, the pharmaceutical formulation comprises an FcRn antagonist in an amount from 300 mg to 6000 mg. In some embodiments, the pharmaceutical formulation comprises an FcRn antagonist in an amount from 750 mg to 3000 mg. In some embodiments, the pharmaceutical formulation comprises an FcRn antagonist in an amount from 1000 mg to 2500 mg. In some embodiments, the pharmaceutical formulation comprises an FcRn antagonist in an amount from 1000 mg to 2000 mg.

In some embodiments, the pharmaceutical formulation comprises about 1000 mg or about 2000 mg of an FcRn antagonist. In some embodiments, the pharmaceutical formulation comprises 1000 mg or 2000 mg of an FcRn antagonist. In some embodiments, the FcRn antagonist is efgartigimod.

In some embodiments, the pharmaceutical formulation comprises efgartigimod in an amount from about 800 mg to about 1200 mg. In some embodiments, the pharmaceutical formulation comprises efgartigimod in an amount from 800 mg to 1200 mg.

In some embodiments, the pharmaceutical formulation comprises about 1000 mg efgartigimod. In some embodiments, the pharmaceutical formulation comprises 1000 mg efgartigimod.

In some embodiments, the pharmaceutical formulation comprises from about 10 mg/mL to about 200 mg/mL efgartigimod. In some embodiments, the pharmaceutical formulation comprises from 10 mg/mL to 200 mg/mL efgartigimod.

In some embodiments, the pharmaceutical formulation comprises about 20 mg/ml efgartigimod. In some embodiments, the pharmaceutical formulation comprises 20 mg/ml efgartigimod.

In some embodiments, the pharmaceutical formulation comprises about 180 mg/mL efgartigimod. In some embodiments, the pharmaceutical formulation comprises 180 mg/mL efgartigimod.

In some embodiments, the pharmaceutical formulation further comprises hyaluronidase. In some embodiments, the hyaluronidase is recombinant human hyaluronidase PH20(rHuPH20).

The hyaluronidase can be present in the pharmaceutical formulation in any suitable amount. In an embodiment, the amount of hyaluronidase enzyme is from about 1000 U/mL to about 3000 U/mL. In an embodiment, the amount of hyaluronidase enzyme is about 1000 U/mL, about 1500 U/mL, about 2000 U/mL, about 2500 U/mL, or about 3000 U/mL. In an embodiment, the amount of hyaluronidase enzyme is 2000 U/mL.

In some embodiments, the rHuPH20 is present in the pharmaceutical formulation in an amount of about 11,000 U. In some embodiments, the rHuPH20 is present in the pharmaceutical formulation in an amount of 11,000 U.

In some embodiments, the pharmaceutical formulation comprises at least about 5 U to at least about 100,000 U of an endoglycosidase hydrolase enzyme. In some aspects, the pharmaceutical formulation comprises at least about 5 U, at least about 10 U, at least about 20 U, at least about 30 U, at least about 40 U, at least about 50 U, at least about 75 U, at least about 100 U, at least about 200 U, at least about 300 U, at least about 400 U, at least about 500 U, at least about 750 U, at least about 1000 U, at least about 2000 U, at least about 3000 U, at least about 4000 U, at least about 5000 U, at least about 6000 U, at least about 7000 U, at least about 8000 U, at least about 9000 U, at least about 10,000 U, at least about 20,000 U, at least about 30,000 U, at least about 40,000 U, at least about 50,000 U, at least about 60,000 U, at least about 70,000 U, at least about 80,000 U, at least about 90,000 U, or at least about 100,000 U of an endoglycosidase hydrolase enzyme.

In some embodiments, the pharmaceutical formulation comprises about 20,000 U of an endoglycosidase hydrolase enzyme. In some embodiments, the pharmaceutical formulation comprises at least about 500 U/mL to at least about 5000 U/mL of an endoglycosidase hydrolase enzyme. In some embodiments, the pharmaceutical formulation comprises at least about 1500 U/mL, at least about 1600 U/mL, at least about 1700 U/mL, at least about 1800 U/mL, at least about 1900 U/mL, at least about 2000 U/mL, at least about 2100 U/mL, at least about 2200 U/mL, at least about 2300 U/mL, at least about 2400 uM, at least about 2500 uM, at least about 3000 uM, at least about 3500 uM, at least about 4000 uM, at least about 4500 U/mL, or at least about 5000 U/mL of an endoglycosidase hydrolase enzyme. In some embodiments, the pharmaceutical formulation comprises about 2000 U/mL of an endoglycosidase hydrolase enzyme.

In some embodiments, the endoglycosidase hydrolase enzyme cleaves hyaluronic acid at a hexosaminidic β (1-4) or (1-3) linkage. In some embodiments, the endoglycosidase hydrolase enzyme comprises a catalytic domain of hyaluronidase PH-20(HuPH20), HYAL1, HYAL2, HYAL3, HYAL4, or HYALPS1. In some embodiments, the endoglycosidase hydrolase enzyme comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to amino acids 36-490 of SEQ ID NO: 32. In some embodiments, the endoglycosidase hydrolase enzyme comprises a hyaluronidase. In some embodiments, the endoglycosidase hydrolase enzyme comprises a hyaluronidase selected from the group consisting of HuPH20, HYAL1, HYAL2, HYAL3, HYAL4, any variant, and any isoform thereof. In some embodiments, the endoglycosidase hydrolase enzyme comprises rHuPH20 or a fragment thereof.

In some embodiments, the endoglycosidase hydrolase enzyme comprises a modified hyaluronidase comprising one or more amino acid substitutions relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in an alpha-helix region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase comprising one or more amino acid substitution in linker region relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified hyaluronidase, wherein one or more N-terminal and/or C-terminal amino acids are deleted relative to a wild-type hyaluronidase selected from the group consisting of HuPH20, HYAL1, HYAL2, HYAL3, HYAL4, HYALPS1, or a fragment thereof. In some embodiments, the endoglycosidase hydrolase enzyme comprises a modified rHuPH20, wherein the modified rHuPH20 comprises: i. one or more amino acid substitution in an alpha-helix region, a linker region, or both an alpha-helix region and a linker region relative to wild-type rHuPH20; ii. deletion of one or more N-terminal amino acid, one or more C-terminal amino acid, or one or more N-terminal amino acid and one or more C-terminal amino acid relative to wild-type rHuPH20; or iii. both (i) and (ii).

“Hyaluronidase,” as used herein, refers to an enzyme capable of catalyzing the cleavage of hyaluronan. Hyaluronan is a repeating polymer of N-acetyl-glucosamine and glucuronic acid, which is present in the subcutaneous space and contributes to the soluble gel-like component of the extracellular matrix of the skin and is restored by rapid turnover (resynthesis). In some embodiments, the hyaluronidase comprises rHuPH20, which is a glycosylated 447-amino acid single chain polypeptide that depolymerizes hyaluronan in the subcutaneous space locally at the site of injection in the skin. Depolymerization of hyaluronan by hyaluronidase is accomplished by hydrolysis of the polysaccharide polymer. Depolymerization of hyaluronan results in a transient reduction in the viscosity of the gel-like phase of the extracellular matrix and increased hydraulic conductance that facilitates the dispersion and absorption of the coadministered therapeutic agent. Thus, a hyaluronidase, e.g., rHuPH20, can improve the speed and ease of subcutaneous delivery of injectable biologics and drugs by acting as a permeation enhancer. In certain embodiments, the hyaluronidase comprises ENHANZE™.

In any of the above embodiments, the pharmaceutical formulation may be a unit dosage form.

In an embodiment, the unit dosage form comprises the FcRn antagonist as a dry formulation for dissolution such as a lyophilized powder, freeze-dried powder, or water-free concentrate. In an embodiment, the dry formulation is comprised in a hermetically sealed container such as a vial, an ampoule, or a sachet.

In an embodiment, the unit dosage form comprises the FcRn antagonist as a liquid formulation, (e.g., injection or infusion solution). In an embodiment, the liquid formulation is comprised in a hermetically sealed container such as a vial, a sachet, a pre-filled syringe, a pre-filled autoinjector, or a cartridge for a reusable syringe or applicator.

In an embodiment, the unit dosage per vial may contain 0.5 ml, 1 ml, 2 ml, 3 ml, 4 ml, 5 ml, 6 ml, 7 ml, 8 ml, 9 ml, 10 ml, 15 ml, or 20 ml of an FcRn antagonist ranging from about 500 to about 2500 mg or from about 1000 mg to about 2000 mg. In an embodiment, these preparations can be adjusted to a desired concentration by adding a sterile diluent to each vial.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist of the invention or other prophylactic or therapeutic agent), and a pharmaceutically acceptable carrier. In an embodiment, the pharmaceutical compositions are formulated to be suitable for subcutaneous administration to a subject.

### Methods

In an aspect, methods for treating pSS using an FcRn antagonist are provided. In certain embodiments, the FcRn antagonist is efgartigimod. An important goal and feature of the methods disclosed herein is improvement in one or more symptoms in pSS patients. Symptoms in pSS patients include, but are not limited to, dryness of mouth and eyes, dryness of nose, sinuses, ears, throat, skin, vagina, and/or prostate, fatigue, and musculoskeletal pain.

pSS can also affect the joints, muscles, nervous system (central nervous system and peripheral nervous system, including the autonomic nervous system), gastrointestinal tract (including the pancreas and liver), skin, blood vessels, lungs, and kidneys. Joint pain and stiffness with mild swelling is common, even in those without rheumatoid arthritis. Rashes may occur, including inflammation of small blood vessels (vasculitis), most commonly on the lower legs. Sun-sensitive rash is more common on the back, chest, face, and arms. Peripheral neuropathy can cause numbness and tingling, especially in the feet, and can frequently pre-date symptoms of dryness. Fatigue, cognitive dysfunction, and sleep abnormalities are frequently reported symptoms.

Other symptoms in pSS patients include cutaneous lesions (e.g., cutaneous vasculitis, annular erythema), pulmonary disorders (e.g., large and/or small airway disease, bronchial/bronchiolar disease), vascular disorders (e.g., Reynaud's phenomenon), renal disease (e.g., glomerulonephritis, tubulointerstitial nephritis, cryoglobulinemia, proteinuria), neurological disorders (e.g., pure sensory neuropathy), autonomic symptoms (e.g., orthostatic intolerance, vasomotor, secretomotor, gastrointestinal, bladder, and pupillomotor symptoms), and/or hematological abnormalities (e.g., cytopenia, raised erythrocyte sedimentation rate, anemia, leucopenia, thrombocytopenia, hypergammaglobulinemia).

Lymphadenopathy, fever, and lymphoma are also complications commonly observed in pSS patients.

Other goals and features of the methods disclosed herein include, but are not limited to, reduced dryness, reduced fatigue, reduced pain, improvement in tear gland function, improvement in salivary gland function, and improvement in disease-related quality of life. Effective treatment of pSS using an FcRn antagonist may include at least one of the elements of the group consisting of: improvement in one or more of the following: Composite of Relevant Endpoints for Sjögren's Syndrome (CRESS), EULAR Sjögren's Syndrome Disease Activity Index (ESSDAI), clinical ESSDAI (clinESSDAI), EULAR Sjögren's Syndrome Patient Reported Index (ESSPRI), Sjögren's Tool for Assessing Response (STAR), Stimulated Whole Salivary Flow (SWSF), Unstimulated Whole Salivary Flow (UWSF), Salivary Gland Ultrasonography (SGUS), Schirmer's Test, Ocular Staining Score (OSS), Hocevar score.

Effective treatment of pSS using an FcRn antagonist may include change in the relative counts of lymphocytic infiltrate (optionally stained for CD45) in the parotid gland and/or change in B/B+T cell ratio in the parotid gland. Effective treatment of pSS using an FcRn antagonist may also include changes in immunophenotype (e.g., plasma cells (IgA, IgG, and IgM)), focus score, number of germinal centers, lymphoepithelial lesions, and/or gene expression per mm² in parotid gland parenchyma. Gene expression may be determined using RNA sequencing and may include genes relevant to immune status and related pathways including, but not limited to, the IFN pathway.

Effective treatment may also include at least one of the elements of the group consisting of: change in serum cytokine/chemokine profiles (including, but not limited to, BAFF or type 1 IFN), reduction in serum C1q immune complexes, reduction in serum IgA, IgM, or IgG-related autoantibodies, reduction in serum IgG, reduction in serum anti-Ro/SS-A, reduction in serum anti-La/SS-B, reduction in serum rheumatoid factor (RF), reduction in serum markers of complement activation (including, but not limited to, C3, C4, and/or split products thereof), change in immunophenotyping in peripheral blood, change in gene expression profile in blood, change in salivary proteins.

Effective treatment may also include at least one of the elements of the group consisting of: improvement in one or more of the following: Multidimensional Fatigue Inventory (MFI), Patient Global Assessment (PGA), 36-Item Short Form Survey (SF-36), EQ-5D-5L, Visual Analog Scale (VAS), Patient Acceptable Symptom State (PASS).

The CRESS composite measures systemic disease activity, patient-reported symptoms, tear gland function, salivary gland function, and serology, developed to assess treatment efficacy in participants with pSS. Use of CRESS to assess pSS disease activity can be advantageous over other measures that report on a single aspect of this heterogeneous disease. CRESS consists of the following items, with definitions of treatment response and lower disease activity:

I Systemic disease activity: as measured with clinESSDAI (see below). Response is defined as a score of <5 points.
Patient-reported symptoms: as measured with ESSPRI (see below). Response is defined as a decrease of ≥1 point or ≥15% from baseline.
Tear gland function: as measured with Schirmer's test and OSS (see below). If Schirmer's test is ≤5 mm at baseline (abnormal), a response is defined as an increase of at least 5 mm from baseline, or if OSS is >3 points at baseline (abnormal), a response is defined as a decrease of at least 2 points from baseline, or if both OSS and Schirmer's scores are normal at baseline, a response is defined as no change that results in an abnormal OSS or Schirmer's score.

Salivary gland function: as measured with UWSF and SGUS (see below). A response is defined as at least 25% increase in UWSF score, or if UWSF score is 0 mL/min at baseline, any increase from baseline, or at least 25% decrease from baseline in total Hocevar score measured using SGUS.

Serology: as measured with serum IgG and RF. A response is defined as a decrease in serum RF of at least 25% from baseline, or a decrease in serum IgG of at least 10% from baseline.

The ESSDAI measures disease activity in patients with pSS. The ESSDAI consists of 12 domains, 11 related to organ involvement (cutaneous, pulmonary, renal, articular, muscular, peripheral nervous system, central nervous system, hematological, glandular, constitutional, and lymphadenopathic) and 1 biological domain reflecting B-cell activity. The activity levels of each domain (range: 0-3 points) are multiplied by their respective weights (range: 1-6 points) to obtain the total score. (See Table S4). Total score ranges from 0-123 points. A score of <5 indicates low disease activity; a score of 5-13 indicates moderate disease activity; a score of ≥14 indicates high disease activity.

ClinESSDAI derives from the ESSDAI, and its score provides an accurate evaluation of disease activity independent of B-cell biomarkers. The clinical domains in clinESSDAI have different weights than in ESSDAI (See Table S5). Total score ranges from 0-135 points. A score of <5 indicates low disease activity; a score of 5-13 indicates moderate disease activity; a score of ≥14 indicates high disease activity.

STAR assesses the efficacy of treatments for pSS. A response is defined as a STAR score of ≥5. This composite measure contains 5 domains:
Systemic activity (3 points): clinESSDAI decrease of ≥3 points;
Patient-reported outcome (3 points): ESSPRI decrease of at least 1 point or ≥15%; symptoms of dryness, pain, and fatigue rated on 3 numeric rating scales;
Lacrimal gland function (1 point; assessed by Schirmer's test or OSS): Schirmer's test
   if abnormal score at baseline: increase of ≥5 mm from baseline; if normal score at baseline: no change to abnormal; OSS-if abnormal score at baseline: decrease of ≥2 points from baseline; if normal score at baseline: no change to abnormal;
Salivary gland function (1 point): UWSF-if score >0 at baseline: increase of ≥25% from baseline; if score is 0 at baseline: any increase in UWSF from baseline; or SGUS >25% decrease in total Hocevar score from baseline;
Biological (1 point; assessed by IgG or RF): IgG: ≥10% reduction or RF: ≥25% decrease.

ESSPRI is a questionnaire that measures self-reported symptoms in participants with pSS. The ESSPRI has 3 items that measure dryness, fatigue, and pain over a recall period of “the last 2 weeks.” Each item includes a numeric rating scale ranging from 0 “No symptoms (dryness, fatigue, or pain)” to 10 “Maximal imaginable (dryness, fatigue, or pain).” The total global score ranges from 0 to 10 and the ESSPRI is calculated by averaging the numeric scores for pain, fatigue, and dryness, with higher scores indicating more symptoms. It has been shown to correlate well with PGA and has been validated in participants with pSS.

The MFI is a 20-item scale designed to evaluate 5 dimensions of fatigue: general fatigue, physical fatigue, reduced motivation, reduced activity, and mental fatigue. Participants report their fatigue over a recall period of “lately” (past 7 days). Each item in the MFI includes 5 boxes ranging from “yes, that is true” to “no, that is not true,” and participants indicate how true certain statements are in regard to their experience with fatigue. Scores in each domain range from 4 to 20, with lower scores corresponding to better health status.

PGA is a tool that measures a participant's global evaluation of their overall disease activity at the time of assessment. The participant rates their overall disease activity by drawing a vertical mark on a 10-cm VAS from the left end of the line (no evidence of disease activity) to the right end of the line (extremely active or severe disease activity).

The SF-36 is a 36-item scale constructed to survey health-related quality of life on 8 domains: limitations in physical activities because of health problems, limitations in social activities because of physical or emotional problems, limitations in usual role activities because of physical health problems, bodily pain, general mental health (psychological distress and well-being), limitations in usual role activities because of emotional problems, vitality (energy and fatigue), and general health perceptions. The SF-36 includes Yes/No questions in addition to 3 point, 5 point, or 6 point Likert response scales. Participants complete the SF 36 using the recall period of “the last 4 weeks.” SF-36 scores are calculated by domain, and total calculated scores for each domain can range from 0 to 100, with higher scores being equated to better health status. The SF-36 may be scored into 2 summary scores: physical component summary (consisting of physical function, physical role, bodily pain, and general health domain total scores) and mental component summary (consisting of vitality, mental health, role emotional, and social function domain total scores).

PASS is a patient-reported outcome measure that assesses the “value beyond which patients consider themselves well.” PASS measures participant well-being and overall feeling that symptoms are in remission, through a single question that is dependent on the indication. PASS assesses the level of symptoms at which participants with rheumatic diseases consider themselves well.

EQ-5D-5L is a standardized measure of health status. It was developed by the EuroQol Group to provide a simple, generic measure of health status for clinical and economic appraisal. The descriptive system comprises 5 dimensions: mobility, self-care, usual activities, pain/discomfort, and anxiety/depression. Each dimension has 5 levels: no problem, slight problem, moderate problem, severe problem, or extreme problem. The participant indicates their health state by selecting the box next to the most appropriate statement in each of the 5 dimensions using the recall period of “today.” This decision results in a 1-digit number expressing the level selected for that dimension. The digits for 5 dimensions were combined in a 5-digit number describing the respondent's health state. A unique health state is defined by combining 1 level from each of the 5 dimensions. A total of 3125 possible health states could be defined in this way. Each state is referred to in terms of a 5-digit code. For example, state 11111 would indicate no problems in any of the 5 dimensions, and 12345 would indicate no problem with mobility, slight problems with washing or dressing, moderate problems with doing usual activities, severe pain or discomfort, and extreme anxiety or depression. A VAS is included in the questionnaire. Respondents mark the health status from 0 to 100 on the day the interview is conducted, with a score of 0 corresponding to “the worst health you can imagine” and 100 corresponding to “the best health you can imagine.”

The SGUS Hocevar grading system rates 5 parameters: parenchymal echogenicity, homogeneity, presence of hypoechogenic areas, hypoechogenic reflections, and the clearness of salivary gland borders. The overall ultrasound score is calculated by summation of the grades for the 5 subscores for all 4 major salivary glands. The overall ultrasound score can range from 0 to 48.

Schirmer's test is an assessment of tear gland function in which a strip of filter paper is applied under the eyelid to measure the quantity of tear production. A result of ≤5 mm indicates abnormal tear gland function.

OSS is another assessment of tear gland function. OSS uses lissamine green dye to grade the conjunctiva and fluorescent dye to grade the cornea. A score of ≥3 points indicates abnormal tear gland function.

In some embodiments, the pSS may be characterized as meeting ACR/EULAR criteria. In some embodiments, the pSS may be characterized as meeting ACR/EULAR 2016 criteria. In some embodiments, the pSS may be characterized as meeting ACR/EULAR criteria≤7 years before administration of an FcRn antagonist. In some embodiments, the pSS may be characterized as meeting ACR/EULAR 2016 criteria≤7 years before administration of an FcRn antagonist.

In some embodiments, the pSS may be characterized by one or more of the following symptoms in a subject: daily, persistent, troublesome dry eyes for more than 3 months; recurrent sensation of sand or gravel in the eyes; use of tear substitutes more than 3 times a day; daily feeling of dry mouth for more than 3 months; and/or frequently drinking liquids to aid in swallowing dry food.

In some embodiments, the pSS may be characterized by an ESSDAI of ≥5 in a subject. In some embodiments, the pSS may be characterized by at least one positive domain item from the ESSDAI questionnaire in a subject.

In some embodiments, the pSS may be characterized by one or more of the following symptoms in a subject: labial salivary gland with focal lymphocytic sialadenitis and focus score of ≥1; anti-Ro/SS-A positive; OSS of ≥5(or van Bijsterfeld score of ≥4) on at least one eye; Schirmer's test of ≤5 mm/5 minutes on at least one eye; and/or UWSF rate of ≤0.1 mL/min.

In some embodiments, the pSS may be characterized by a score of ≥4 when summing the weights of the items presented in Table S1.

In some embodiments, the pSS may be characterized by presence of autoantibodies in a subject. In some embodiments, the autoantibody may be one or more of an anti-Ro/SS-A autoantibody, an anti-La/SS-B autoantibody, an ANA autoantibody, or rheumatoid factor (RF). In some embodiments, the pSS may be characterized by presence of anti-Ro/SS-A autoantibodies in a subject. In some embodiments, the presence of autoantibodies is detected in serum from the subject.

In some embodiments, the pSS may be characterized by residual salivary flow in a subject. In some embodiments, the pSS may be characterized by a UWSF rate of >0 in a subject. In some embodiments, the pSS may be characterized by a SWSF rate>0.10 in a subject. In some embodiments, the pSS may be characterized by a UWSF rate of >0 and a SWSF rate of >0.10 in a subject.

In some embodiments, the pSS may be characterized by serum total IgG >6 g/L in a subject. In some embodiments, the pSS may be characterized by serum total IgG >4 g/L in a subject.

In some embodiments, the subject has at least a moderate level of systemic disease activity.

In some embodiments, the subject has not been diagnosed with, or received treatment for, one or more of the following conditions: AIDS, active hepatitis C infection, sarcoidosis, amyloidosis, graft-versus-host disease, history of head and neck radiation treatment, or IgG4-related disease. In some embodiments, the subject has not been diagnosed with, or received treatment for, any of the following conditions: AIDS, active hepatitis C infection, sarcoidosis, amyloidosis, graft-versus-host disease, history of head and neck radiation treatment, or IgG4-related disease.

In some embodiments, the subject has not been diagnosed with, or received treatment for, a secondary SS overlap syndrome where another confirmed autoimmune rheumatic or systemic inflammatory condition is the primary diagnosis. In some embodiments, the subject has not received a primary diagnosis of one or more of the following conditions: rheumatoid arthritis, systemic lupus erythematosus, scleroderma, or inflammatory bowel disease. In some embodiments, the subject has not received a primary diagnosis of any of the following conditions: rheumatoid arthritis, systemic lupus erythematosus, scleroderma, or inflammatory bowel disease.

In some embodiments, the subject has no history of malignancy unless considered cured by adequate treatment with no evidence of recurrence for >3 years before the first administration of IMP. In some embodiments, subjects do have basal cell or squamous cell skin cancer, carcinoma in situ of the cervix, carcinoma in situ of the breast, or incidental histological finding of prostate cancer (TNM stage T1a or T1b).

In some embodiments, the subject does not have significant uncontrolled active or chronic bacterial, viral, or fungal infection, or positive SARS-COV-2 PCR test. In some embodiments, the subject has no positive serum test for hepatitis B virus (HBV) that is indicative of an acute or chronic infection, unless associated with a negative HB surface antigen (HBsAg) or negative HBV DNA test, hepatitis C virus (HCV) based on HCV antibody assay unless a negative RNA test is available, or HIV. In some embodiments, the subject does not have total IgG <4 g/L.

In some embodiments, the subject has not been treated with a biologic disease-modifying antirheumatic drug (DMARD) prior to administration of an FcRn antagonist. In some embodiments, the subject has not been treated with a biologic DMARD within 24 weeks prior to administration of an FcRn antagonist.

In some embodiments, the subject has not been treated with one or more of the following: intravenous immunoglobulin (IVIg), subcutaneous immunoglobulin (SCIg), or plasma exchange (PLEX) prior to administration of an FcRn antagonist. In some embodiments, the subject has not been treated with any of the following: IVIg, SCIg, or PLEX prior to administration of an FcRn antagonist. In some embodiments, the subject has not been treated with one or more of the following: IVIg, SCIg, or PLEX within 12 weeks prior to administration of an FcRn antagonist. In some embodiments, the subject has not been treated with any of the following: IVIg, SCIg, or PLEX within 12 weeks prior to administration of an FcRn antagonist.

In some embodiments, the subject has not been treated with one or more of the following: a pharmacological stimulant for salivary or lacrimal glands, pilocarpine, systemic corticosteroids, antimalarials, conventional DMARDs, or JAK inhibitors prior to administration of an FcRn antagonist. In some embodiments, the subject has not been treated with any of the following: a pharmacological stimulant for salivary or lacrimal glands, pilocarpine, systemic corticosteroids, antimalarials, conventional DMARDs, or JAK inhibitors prior to administration of an FcRn antagonist. In some embodiments, the subject has not been treated with one or more of the following: a pharmacological stimulant for salivary or lacrimal glands, pilocarpine, systemic corticosteroids, antimalarials, conventional DMARDs, or JAK inhibitors within 4 weeks prior to administration of an FcRn antagonist. In some embodiments, the subject has not been treated with any of the following: a pharmacological stimulant for salivary or lacrimal glands, pilocarpine, systemic corticosteroids, antimalarials, conventional DMARDs, or JAK inhibitors within 4 weeks prior to administration of an FcRn antagonist.

In some embodiments, the FcRn antagonist is administered at a fixed dose of about 20 mg to about 20,000 mg. In some embodiments, the FcRn antagonist is administered at a fixed dose of about 200 mg to about 20,000 mg. In some embodiments, the FcRn antagonist is administered at a fixed dose of about 300 mg to about 6000 mg. In some embodiments, the FcRn antagonist is administered at a fixed dose of about 750 mg to about 3000 mg. In some embodiments, the FcRn antagonist is administered at a fixed dose of about 1000 mg to about 2500 mg. In some embodiments, the FcRn antagonist is administered at a fixed dose of about 1000 mg to about 2000 mg. In some embodiments, the FcRn antagonist is efgartigimod.

In some embodiments, the FcRn antagonist is administered at a fixed dose of 20 mg to 20,000 mg. In some embodiments, the FcRn antagonist is administered at a fixed dose of 200 mg to 20,000 mg. In some embodiments, the FcRn antagonist is administered at a fixed dose of 300 mg to 6000 mg. In some embodiments, the FcRn antagonist is administered at a fixed dose of 750 mg to 3000 mg. In some embodiments, the FcRn antagonist is administered at a fixed dose of 1000 mg to 2500 mg. In some embodiments, the FcRn antagonist is administered at a fixed dose of 1000 mg to 2000 mg. In some embodiments, the FcRn antagonist is efgartigimod.

In some embodiments, the FcRn antagonist is administered at a fixed dose of about 20 mg, about 50 mg, about 100 mg, about 200 mg, about 250 mg, about 300 mg, about 500 mg, about 750 mg, about 1000 mg, about 1500 mg, about 2000 mg, about 2500 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg. In some embodiments, the FcRn antagonist is efgartigimod.

In some embodiments, the FcRn antagonist is administered at a fixed dose of 20 mg, 50 mg, 100 mg, 200 mg, 250 mg, 300 mg, 500 mg, 750 mg, 1000 mg, 1500 mg, 2000 mg, 2500 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg. In some embodiments, the FcRn antagonist is efgartigimod.

In some embodiments, the FcRn antagonist is administered at a dose of about 0.2 mg/kg to about 200 mg/kg. In some embodiments, the FcRn antagonist is administered at a dose of about 2 mg/kg to about 200 mg/kg. In some embodiments, the FcRn antagonist is administered at a dose of about 2 mg/kg to about 120 mg/kg. In some embodiments, the FcRn antagonist is administered at a dose of about 3 mg/kg to about 60 mg/kg. In some embodiments, the FcRn antagonist is administered at a dose of about 10 mg/kg to about 25 mg/kg. In some embodiments, the FcRn antagonist is efgartigimod.

In some embodiments, the FcRn antagonist is administered at a dose of 0.2 mg/kg to 200 mg/kg. In some embodiments, the FcRn antagonist is administered at a dose of about 2 mg/kg to about 200 mg/kg. In some embodiments, the FcRn antagonist is administered at a dose of 2 mg/kg to 120 mg/kg. In some embodiments, the FcRn antagonist is administered at a dose of 3 mg/kg to 60 mg/kg. In some embodiments, the FcRn antagonist is administered at a dose of 10 mg/kg to 25 mg/kg. In some embodiments, the FcRn antagonist is efgartigimod.

In some embodiments, the FcRn antagonist is administered at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously. In some embodiments, the FcRn antagonist is administered intravenously once weekly, once every two weeks, once every three weeks, once every four weeks, once monthly, or once every six weeks. In some embodiments, the FcRn antagonist is efgartigimod.

In some embodiments, the FcRn antagonist is administered intravenously once weekly or once every two weeks at a dose of about 0.2 mg/kg to about 200 mg/kg. In some embodiments, the FcRn antagonist is administered intravenously once weekly or once every two weeks at a dose of about 2 mg/kg to about 200 mg/kg. In some embodiments, the FcRn antagonist is administered intravenously once weekly or once every two weeks at a dose of about 2 mg/kg to about 120 mg/kg. In some embodiments, the FcRn antagonist is administered intravenously once weekly or once every two weeks at a dose of about 3 mg/kg to about 60 mg/kg. In some embodiments, the FcRn antagonist is administered intravenously once weekly or once every two weeks at a dose of about 10 mg/kg to about 25 mg/kg. In some embodiments, the FcRn antagonist is efgartigimod.

In some embodiments, the FcRn antagonist is administered intravenously once weekly or once every two weeks at a dose of 0.2 mg/kg to 200 mg/kg. In some embodiments, the FcRn antagonist is administered intravenously once weekly or once every two weeks at a dose of 2 mg/kg to 200 mg/kg. In some embodiments, the FcRn antagonist is administered intravenously once weekly or once every two weeks at a dose of 2 mg/kg to 120 mg/kg. In some embodiments, the FcRn antagonist is administered intravenously once weekly or once every two weeks at a dose of 3 mg/kg to 60 mg/kg. In some embodiments, the FcRn antagonist is administered intravenously once weekly or once every two weeks at a dose of 10 mg/kg to 25 mg/kg. In some embodiments, the FcRn antagonist is efgartigimod.

In some embodiments, the FcRn antagonist is administered intravenously once weekly or once every two weeks at a dose of about 0.2 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12.5 mg/kg, about 15 mg/kg, about 17.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, or about 200 mg/kg. In some embodiments, the FcRn antagonist is efgartigimod.

In some embodiments, the FcRn antagonist is administered intravenously once weekly or once every two weeks at a dose of 0.2 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, or 200 mg/kg. In some embodiments, the FcRn antagonist is efgartigimod.

In some embodiments, the FcRn antagonist is administered intravenously once weekly or once every two weeks at a dose of about 10 mg/kg to about 30 mg/kg. In some embodiments, the FcRn antagonist is administered intravenously once weekly or once every two weeks at a dose of about 10 mg/kg to about 25 mg/kg. In some embodiments, the FcRn antagonist is administered intravenously once weekly or once every two weeks at a dose of about 10 mg/kg. In some embodiments, the FcRn antagonist is administered intravenously once weekly or once every two weeks at a dose of about 15 mg/kg. In some embodiments, the FcRn antagonist is administered intravenously once weekly or once every two weeks at a dose of about 20 mg/kg. In some embodiments, the FcRn antagonist is administered intravenously once weekly or once every two weeks at a dose of about 25 mg/kg. In some embodiments, the FcRn antagonist is administered intravenously once weekly or once every two weeks at a dose of about 30 mg/kg. In some embodiments, the FcRn antagonist is administered intravenously once weekly or once every two weeks at a dose of 10 mg/kg to 30 mg/kg. In some embodiments, the FcRn antagonist is administered intravenously once weekly or once every two weeks at a dose of 10 mg/kg to 25 mg/kg. In some embodiments, the FcRn antagonist is administered intravenously once weekly or once every two weeks at a dose of 10 mg/kg. In some embodiments, the FcRn antagonist is administered intravenously once weekly or once every two weeks at a dose of 15 mg/kg. In some embodiments, the FcRn antagonist is administered intravenously once weekly or once every two weeks at a dose of 20 mg/kg. In some embodiments, the FcRn antagonist is administered intravenously once weekly or once every two weeks at a dose of 25 mg/kg. In some embodiments, the FcRn antagonist is administered intravenously once weekly or once every two weeks at a dose of 30 mg/kg. In some embodiments, the FcRn antagonist is efgartigimod.

In some embodiments, the FcRn antagonist is administered intravenously once every two weeks for 52 weeks. In some embodiments, the FcRn antagonist is efgartigimod.

In some embodiments, the FcRn antagonist is administered subcutaneously. In some embodiments, the FcRn antagonist is administered subcutaneously once weekly, once every two weeks, once every three weeks, once every four weeks, once monthly, or once every six weeks. In some embodiments, the FcRn antagonist is efgartigimod.

In some embodiments, FcRn antagonist is administered subcutaneously at a fixed dose of about 20 mg to about 20,000 mg. In some embodiments, FcRn antagonist is administered subcutaneously at a fixed dose of about 100 mg to about 10,000 mg once weekly, once every two weeks, once every three weeks, once every four weeks, once monthly, or once every six weeks. In some embodiments, the FcRn antagonist is administered subcutaneously at a fixed dose of 750 mg to 3000 mg once weekly, once every two weeks, once every three weeks, once every four weeks, once monthly, or once every six weeks. In some embodiments, the FcRn antagonist is administered subcutaneously at a fixed dose of 1000 mg to 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of about 20 mg, about 50 mg, about 100 mg, about 250 mg, about 500 mg, about 750 mg, about 1000 mg, about 1500 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously at a fixed dose of 20 mg, 50 mg, 100 mg, 250 mg, 500 mg, 750 mg, 1000 mg, 1500 mg, 2000 mg, 3000 mg, 4000 mg, 5000 mg, 6000 mg, 7000 mg, 8000 mg, 9000 mg, 10,000 mg, 11,000 mg, 12,000 mg, 13,000 mg, 14,000 mg, 15,000 mg, 16,000 mg, 17,000 mg, 18,000 mg, 19,000 mg, or 20,000 mg once weekly, once every two weeks, once every three weeks, once every four weeks, once monthly, or once every six weeks. In some embodiments, the FcRn antagonist is administered subcutaneously at a fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered subcutaneously once weekly or every two weeks at a fixed dose of about 750 mg to about 3000 mg. In some embodiments, the FcRn antagonist is administered subcutaneously once weekly or every two weeks at a fixed dose of about 1000 mg to about 2000 mg. In some embodiments, the FcRn antagonist is administered subcutaneously at a fixed dose of about 1000 mg or about 2000 mg once weekly or every two weeks. In some embodiments, the FcRn antagonist is efgartigimod.

In some embodiments, the FcRn antagonist is administered subcutaneously once weekly or every two weeks at a fixed dose of 750 mg to 3000 mg. In some embodiments, the FcRn antagonist is administered subcutaneously at a fixed dose of 750 mg to 3000 mg once weekly. In some embodiments, the FcRn antagonist is administered subcutaneously at a fixed dose of 750 mg to 3000 mg once every two weeks. In some embodiments, the FcRn antagonist is administered subcutaneously at a fixed dose of 750 mg to 3000 mg once every three weeks. In some embodiments, the FcRn antagonist is administered subcutaneously at a fixed dose of 750 mg to 3000 mg once monthly. In some embodiments, the FcRn antagonist is administered subcutaneously once weekly or every two weeks at a fixed dose of 1000 mg to 2000 mg. In some embodiments, the FcRn antagonist is administered subcutaneously at a fixed dose of 1000 mg or 2000 mg once weekly or every two weeks. In some embodiments, the FcRn antagonist is efgartigimod.

In some embodiments, the FcRn antagonist is first administered subcutaneously at a fixed dose of about 1000 mg twice on the same day. In some embodiments, the FcRn antagonist is first administered subcutaneously at a fixed dose of 1000 mg twice on the same day. In some embodiments, the FcRn antagonist is efgartigimod.

In some embodiments, the FcRn antagonist is administered subcutaneously once weekly at a fixed dose of about 750 mg to about 1750 mg. In some embodiments, the FcRn antagonist is administered subcutaneously once weekly at a fixed dose of about 800 mg to about 1200 mg. In some embodiments, the FcRn antagonist is administered subcutaneously once weekly at a fixed dose of about 750 mg. In some embodiments, the FcRn antagonist is administered subcutaneously once weekly at a fixed dose of about 800 mg. In some embodiments, the FcRn antagonist is administered subcutaneously once weekly at a fixed dose of about 1000 mg. In some embodiments, the FcRn antagonist is administered subcutaneously once weekly at a fixed dose of about 1200 mg. In some embodiments, the FcRn antagonist is administered subcutaneously once weekly at a fixed dose of about 1250 mg. In some embodiments, the FcRn antagonist is administered subcutaneously once weekly at a fixed dose of about 1500 mg. In some embodiments, the FcRn antagonist is administered subcutaneously once weekly at a fixed dose of about 1750 mg. In some embodiments, the FcRn antagonist is efgartigimod.

In some embodiments, the FcRn antagonist is administered subcutaneously once weekly at a fixed dose of 750 mg to 1750 mg. In some embodiments, the FcRn antagonist is administered subcutaneously once weekly at a fixed dose of 800 mg to 1200 mg. In some embodiments, the FcRn antagonist is administered subcutaneously once weekly at a fixed dose of 750 mg. In some embodiments, the FcRn antagonist is administered subcutaneously once weekly at a fixed dose of 800 mg. In some embodiments, the FcRn antagonist is administered subcutaneously once weekly at a fixed dose of 1000 mg. In some embodiments, the FcRn antagonist is administered subcutaneously once weekly at a fixed dose of 1200 mg. In some embodiments, the FcRn antagonist is administered subcutaneously once weekly at a fixed dose of 1250 mg. In some embodiments, the FcRn antagonist is administered subcutaneously once weekly at a fixed dose of 1500 mg. In some embodiments, the FcRn antagonist is administered subcutaneously once weekly at a fixed dose of 1750 mg. In some embodiments, the FcRn antagonist is efgartigimod.

In some embodiments, the FcRn antagonist is administered subcutaneously once weekly at a dose of about 10 mg/kg to about 25 mg/kg. In some embodiments, the FcRn antagonist is administered subcutaneously once weekly at a dose of about 10 mg/kg. In some embodiments, the FcRn antagonist is administered subcutaneously once weekly at a dose of about 15 mg/kg. In some embodiments, the FcRn antagonist is administered subcutaneously once weekly at a dose of about 20 mg/kg. In some embodiments, the FcRn antagonist is administered subcutaneously once weekly at a dose of about 25 mg/kg. In some embodiments, the FcRn antagonist is efgartigimod.

In some embodiments, the FcRn antagonist is administered subcutaneously once weekly at a dose of 10 mg/kg to 25 mg/kg. In some embodiments, the FcRn antagonist is administered subcutaneously once weekly at a dose of 10 mg/kg. In some embodiments, the FcRn antagonist is administered subcutaneously once weekly at a dose of 15 mg/kg. In some embodiments, the FcRn antagonist is administered subcutaneously once weekly at a dose of 20 mg/kg. In some embodiments, the FcRn antagonist is administered subcutaneously once weekly at a dose of 25 mg/kg. In some embodiments, the FcRn antagonist is efgartigimod.

In some embodiments, the FcRn antagonist is first administered intravenously and is subsequently administered subcutaneously. In some embodiments, the FcRn antagonist is first administered intravenously and is subsequently administered subcutaneously at fixed dose of 100 mg to 10,000 mg once weekly, once every two weeks, once every three weeks, once every four weeks, once monthly, or once every six weeks. In some embodiments, the FcRn antagonist is first administered intravenously and is subsequently administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, one or more doses of the FcRn antagonist are administered intravenously and subsequent doses of the FcRn antagonist are administered subcutaneously. In some embodiments, one or more doses of the FcRn antagonist are administered intravenously and subsequent doses of the FcRn antagonist are administered subcutaneously at fixed dose of 100 mg to 10,000 mg once weekly, once every two weeks, once every three weeks, once every four weeks, once monthly, or once every six weeks. In some embodiments, one or more doses of the FcRn antagonist are administered intravenously and subsequent doses of the FcRn antagonist are administered subcutaneously at fixed dose of 1000 mg or 2000 mg once weekly, once every two weeks, once every three weeks, once every four weeks, once monthly, or once every six weeks. In some embodiments, the FcRn antagonist is efgartigimod.

In some embodiments, the FcRn antagonist is administered for 6, 12, 24, 39, or 52 weeks or less. In some embodiments, the FcRn antagonist is administered for 24 weeks or less. In some embodiments, the FcRn antagonist is administered for 52 weeks or less. In some embodiments, the FcRn antagonist is administered for at least 6, 12, 24, 39, or 52 weeks. In some embodiments, the FcRn antagonist is administered for at least 24 weeks. In some embodiments, the FcRn antagonist is administered for at least 52 weeks.

In some embodiments, the FcRn antagonist is rozanolixizumab. In some embodiments, rozanolixizumab is administered subcutaneously or intravenously. In some embodiments, rozanolixizumab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, rozanolixizumab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, rozanolixizumab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is nipocalimab. In some embodiments, nipocalimab is administered subcutaneously or intravenously. In some embodiments, nipocalimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, nipocalimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, nipocalimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the FcRn antagonist is orilanolimab. In some embodiments, orilanolimab is administered subcutaneously or intravenously. In some embodiments, orilanolimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, orilanolimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, orilanolimab is administered intravenously at a dose of about 30 mg/kg once weekly for three weeks and then at a dose of 10 mg/kg administered intravenously every other week.

In some embodiments, the FcRn antagonist is batoclimab. In some embodiments, batoclimab is administered subcutaneously or intravenously. In some embodiments, batoclimab is administered at a dose of about 0.2 mg/kg to about 200 mg/kg or at a fixed dose of about 20 mg to about 20,000 mg administered once weekly, once every two weeks, once every three weeks, once every four weeks, once monthly, or once every six weeks.

In some embodiments, batoclimab is administered once weekly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every two weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every three weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once every four weeks at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, batoclimab is administered once monthly at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 12.5 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In some embodiments, the method further comprises administering to the subject an additional therapeutic compound.

In some embodiments, the method further comprises administering to the subject an effective amount of an anticholinergic agent.

In some embodiments, the method further comprises administering to the subject a topical medication. In some embodiments, the method further comprises administering to the subject an effective amount of one or more of the following: an ophthalmic lubricant, an ophthalmic lubricating ointment, a hydroxyl cellulose insert, or a saliva substitute. In some embodiments, the method further comprises administering to the subject an effective amount of a pharmacological stimulant for salivary and/or lacrimal glands. In some embodiments, the method further comprises administering to the subject an effective amount of one or more of the following: cyclosporine, lifitegrast, pilocarpine, or cevimeline.

In some embodiments, the method further comprises administering to the subject an effective amount of one or more of the following: a DMARD, a janus kinase (JAK) inhibitor, an NSAID (e.g., ibuprofen, naproxen), acetaminophen, or an antimalarial (e.g., hydroxychloroquine).

In an embodiment, the method further comprises administering to the subject an effective amount of a corticosteroid and/or an immunosuppressive agent. In an embodiment, the method further comprises administering to the subject an effective amount of a corticosteroid. In an embodiment, method further comprises administering to the subject an effective amount of a glucocorticoid. In an embodiment, the method further comprises administering to the subject an effective amount of a glucocorticoid intravenously and/or administering to the subject an effective amount of a glucocorticoid orally. In an embodiment, the method further comprises administering to the subject an effective amount of a glucocorticoid intravenously and administering to the subject an effective amount of a glucocorticoid orally.

In an embodiment, the method further comprises administering to the subject an effective amount of prednisone. In an embodiment, the method further comprises administering to the subject prednisone at a dose of 7.5 mg/day to 75 mg/day, to a maximum of 1 mg/kg/day. In an embodiment, the method further comprises administering to the subject prednisone at a dose of 8 mg/day to 72 mg/day, to a maximum of 1 mg/kg/day. In an embodiment, the method further comprises administering to the subject prednisone at a dose of 9 mg/day to 66 mg/day, to a maximum of 1 mg/kg/day. In an embodiment, the method further comprises administering to the subject prednisone at a dose of 10 mg/day to 60 mg/day, to a maximum of 1 mg/kg/day. In an embodiment, the method further comprises administering to the subject prednisone at a dose of 0.5 mg/kg/day to 1 mg/kg/day. In an embodiment, the method further comprises administering to the subject prednisone at a dose of 0.6 mg/kg/day to 1 mg/kg/day. In an embodiment, the method further comprises administering to the subject prednisone at a dose of 0.6 mg/kg/day to 1 mg/kg/day, to a maximum of 80 mg/day. In an embodiment, the prednisone is administered orally.

In an embodiment, the method further comprises administering to the subject an effective amount of methylprednisolone. In an embodiment, the method further comprises administering to the subject methylprednisolone at a dose of 100 mg to 1250 mg, for up to three days. In an embodiment, the method further comprises administering to the subject methylprednisolone at a dose of 150 mg to 1200 mg, for up to three days. In an embodiment, the method further comprises administering to the subject methylprednisolone at a dose of 200 mg to 1100 mg, for up to three days. In an embodiment, the method further comprises administering to the subject methylprednisolone at a dose of 500 mg to 1000 mg, for up to three days. In an embodiment, the method further comprises administering to the subject methylprednisolone at a dose of 0.25 g/day to 0.5 g/day. In an embodiment, the method further comprises administering to the subject methylprednisolone at a dose of 0.25 g/day to 0.5 g/day, for one to three days. In an embodiment, the methylprednisolone is administered intravenously.

In an embodiment, the method further comprises administering to the subject an effective amount of prednisone orally and an effective amount of methylprednisolone intravenously. In an embodiment, the method further comprises administering to the subject prednisone orally at a dose of 10 mg/day to 60 mg/day, to a maximum of 1 mg/kg/day, and methylprednisolone intravenously at a dose of 500 mg to 1000 mg, for up to three days. In an embodiment, the method further comprises administering to the subject prednisone orally at a dose of 0.5 mg/kg/day to 1 mg/kg/day, and methylprednisolone intravenously at a dose of 500 mg to 1000 mg, for up to three days. In an embodiment, the method further comprises administering to the subject prednisone orally at a dose of 0.6 mg/kg/day to 1 mg/kg/day, to a maximum of 80 mg/day, and methylprednisolone intravenously at a dose of 0.25 g/day to 0.5 g/day, for one to three days.

In an embodiment, the method further comprised administering to the subject an effective amount of an immunosuppressive agent. Examples of immunosuppressive agents include, without limitation, methotrexate, azathioprine, mycophenolate mofetil, leflunomide, and cyclophosphamide.

In an embodiment, the method further comprises administering to the subject an effective amount of a B-lymphocyte targeting biologic. Examples of B-lymphocyte targeting biologics include, without limitation, belimumab, rituximab, and obinutuzumab. In an embodiment, the method further comprises administering to the subject an effective amount of belimumab. In an embodiment, the method further comprises administering to the subject belimumab intravenously at a dose of 10 mg/kg once every two weeks for three doses, then once every four weeks for subsequent doses. In an embodiment, the method further comprises administering to the subject belimumab intravenously at a dose of 10 mg/kg once every two weeks for three doses, then once every four weeks for subsequent doses, and a mycophenolic acid analog. In an embodiment, the method further comprises administering to the subject belimumab intravenously at a dose of 10 mg/kg once every two weeks for three doses, then once every four weeks for subsequent doses, and cyclophosphamide. In an embodiment, the method further comprises administering to the subject belimumab intravenously at a dose of 10 mg/kg once every two weeks for three doses, then once every four weeks for subsequent doses, and cyclophosphamide at a dose of 500 mg once every two weeks for six months. In an embodiment, the method further comprises administering to the subject an effective amount of rituximab. In an embodiment, the method further comprises administering to the subject rituximab at a dose of 1 g on days 1 and 15 as add-on therapy for refractory cases or for corticosteroid minimization. In an embodiment, the method further comprises administering to the subject an effective amount of obinutuzumab.

In an embodiment, when the subject is administered the FcRn antagonist, the dose of prednisone is tapered over 12 weeks to a dose of 7.5 mg/day. In an embodiment, the initial oral prednisone dose is at 0.5 mg/kg/day to 1 mg/kg/day, not to exceed 60 mg/day.

In some embodiments, treatment of pSS is characterized by the subject exhibiting one or more responses following administration of the FcRn antagonist. In some embodiments, treatment of pSS is characterized by the subject exhibiting two or more responses following administration of the FcRn antagonist. In some embodiments, treatment of pSS is characterized by the subject exhibiting three or more responses following administration of the FcRn antagonist. In some embodiments, treatment of pSS is characterized by the subject exhibiting four or more responses following administration of the FcRn antagonist. In some embodiments, treatment of pSS is characterized by the subject exhibiting five or more responses following administration of the FcRn antagonist. In some embodiments, the responses are selected from the group consisting of: 1) a clinical ESSDAI (clinESSDAI) score of <5 points; 2) a decrease in EULAR Sjögren's syndrome patient reported index (ESSPRI) score of ≥1 point or ≥15%, compared to a baseline value; 3) an increase in tear gland function; 4) an increase in salivary gland function; and 5) a decrease in serum rheumatoid factor (RF) of at least 25%, compared to a baseline value, or a decrease in serum IgG of at least 10%, compared to a baseline value. In some embodiments, the baseline value of any of the above is obtained from the subject prior to administration of the FcRn antagonist. In some embodiments, the baseline value of any of the above is obtained from the subject prior to an initial administration of the FcRn antagonist. In some embodiments, one or more of the responses are measured 16 weeks and/or 24 weeks after administration of an FcRn antagonist. In some embodiments, one or more of the responses are measured 16 weeks and/or 24 weeks after an initial administration of the FcRn antagonist.

In some embodiments, the increase in tear gland function is measured by Schirmer's test and/or ocular staining score (OSS). In some embodiments, if the subject shows a baseline value of ≤5 mm as measured by Schirmer's test, a response is defined as an increase of at least 5 mm from the baseline value. In some embodiments, if the subject shows a baseline value of >3 points as measured by OSS, a response is defined as a decrease of at least 2 points from the baseline value. In some embodiments, if the subject shows a baseline value of >5 mm as measured by Schirmer's test and shows a baseline value of <3 points as measured by OSS, a response is defined as no change that results in an abnormal OSS or Schirmer's score. In some embodiments, the baseline value of any of the above is obtained from the subject prior to administration of the FcRn antagonist. In some embodiments, the baseline value of any of the above is obtained from the subject prior to an initial administration of the FcRn antagonist. In some embodiments, one or more of the responses are measured 16 weeks and/or 24 weeks after administration of the FcRn antagonist. In some embodiments, one or more of the responses are measured 16 weeks and/or 24 weeks after an initial administration of the FcRn antagonist.

In some embodiments, the increase in salivary gland function is measured by UWSF and/or salivary gland ultrasonography (SGUS). In some embodiments, a response is defined as an increase in UWSF in the subject of at least 25%, compared to a baseline value if the baseline value is >0 mL/min, or any increase in UWSF in the subject if the baseline value is 0 mL/min. In some embodiments, a response is defined as a decrease in Hocevar score in the subject as measured by SGUS of at least 25%, compared to a baseline value. In some embodiments, the baseline value of any of the above is obtained from the subject prior to administration of the FcRn antagonist. In some embodiments, the baseline value of any of the above is obtained from the subject prior to an initial administration of the FcRn antagonist. In some embodiments, one or more of the responses are measured 16 weeks and/or 24 weeks after administration of the FcRn antagonist. In some embodiments, one or more of the responses are measured 16 weeks and/or 24 weeks after an initial administration of the FcRn antagonist.

In some embodiments, the subject shows a change in CD45+lymphocytic infiltrate in the parotid gland following administration of the FcRn antagonist, compared to a baseline value. In some embodiments, the subject shows a decrease in CD45+lymphocytic infiltrate in the parotid gland following administration of the FcRn antagonist of at least 5%, at least 10%, at least 15%, at least 20%, at least 25%, at least 30%, at least 40%, at least 50%, at least 60%, at least 70%, at least 80%, or at least 90%, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the CD45+lymphocytic infiltrate in the parotid gland is measured 24 weeks following administration of the FcRn antagonist. In some embodiments, the CD45+lymphocytic infiltrate in the parotid gland is measured 24 weeks following an initial administration of the FcRn antagonist.

In some embodiments, the subject shows a change in B/B+T cell ratio in the parotid gland following administration of the FcRn antagonist, compared to a baseline value. In some embodiments, the subject shows a decrease in B/B+T cell ratio in the parotid gland following administration of the FcRn antagonist of at least 5%, at least 10%, at least 15%, at least 20%, at least 25%, at least 30%, at least 40%, at least 50%, at least 60%, at least 70%, at least 80%, or at least 90%, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the B/B+T cell ratio in the parotid gland is measured 24 weeks following administration of the FcRn antagonist. In some embodiments, the B/B+T cell ratio in the parotid gland is measured 24 weeks following an initial administration of the FcRn antagonist.

In some embodiments, the subject shows a decrease in ESSDAI score, clinESSDAI score, and/or ESSPRI score following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the ESSDAI score, the clinESSDAI score, and/or the ESSPRI score is measured 16 weeks and/or 24 weeks following administration of the FcRn antagonist. In some embodiments, the ESSDAI score, the clinESSDAI score, and/or the ESSPRI score is measured 16 weeks and/or 24 weeks following an initial administration of the FcRn antagonist.

In some embodiments, the subject shows a decrease of at least 1, 2, 3, 4, 5, 10, 15, 20, 25, 30, 35, 40, 45, or 50 points in the ESSDAI score following administration of the FcRn antagonist, compared to a baseline value. In some embodiments, the subject shows a decrease of at least 1, 2, 3, 4, 5, 10, 15, 20, 25, 30, 35, 40, 45, or 50 points in the clinESSDAI score following administration of the FcRn antagonist, compared to a baseline value. In some embodiments, the subject shows a decrease of at least 1, 2, 3, 4, 5, 10, 15, 20, 25, 30, 35, 40, 45, or 50 points in the ESSDAI score and the clinESSDAI score following administration of the FcRn antagonist, compared to a baseline value. In some embodiments, the subject shows a decrease of at least 3 points in the ESSDAI score following administration of the FcRn antagonist, compared to a baseline value. In some embodiments, the subject shows a decrease of at least 3 points in the clinESSDAI score following administration of the FcRn antagonist, compared to a baseline value. In some embodiments, the subject shows a decrease of at least 3 points in the ESSDAI score and the clinESSDAI score following administration of the FcRn antagonist, compared to a baseline value.

In some embodiments, the subject shows a decrease of at least 1, 2, 3, 4, 5, 6, 7, 8, or 9 points in the ESSPRI score following administration of the FcRn antagonist, compared to a baseline value. In some embodiments, the subject shows a decrease of at least 1 point in the ESSPRI score following administration of the FcRn antagonist, compared to a baseline value. In some embodiments, the subject shows a decrease of ≥10%, ≥15%, ≥20%, ≥30%, >40%, ≥50%, >60%, >70%, >80%, or >90% in the ESSPRI score following administration of the FcRn antagonist, compared to a baseline value. In some embodiments, the subject shows a decrease of ≥15% in the ESSPRI score following administration of the FcRn antagonist, compared to a baseline value. In some embodiments, the subject shows a decrease of at least 1 point and a decrease of >15% in the ESSPRI score following administration of the FcRn antagonist, compared to a baseline value.

In some embodiments, the subject has an ESSDAI score of <1, <2, <3, <4, <5, <6, <7, <8, <9, <10, <11, <12, <13, or <14 following administration of the FcRn antagonist. In some embodiments, the subject has a clinESSDAI score of <1, <2, <3, <4, <5, <6, <7, <8, <9, <10, <11, <12, <13, or <14 following administration of the FcRn antagonist. In some embodiments, the subject has an ESSDAI score of <1, <2, <3, <4, <5, <6, <7, <8, <9, <10, <11, <12, <13, or <14 and a clinESSDAI score of <1, <2, <3, <4, <5, <6, <7, <8, <9, <10, <11, <12, <13, or <14 following administration of the FcRn antagonist. In some embodiments, the subject has an ESSDAI score of <5 following administration of the FcRn antagonist. In some embodiments, the subject has a clinESSDAI score of <5 following administration of the FcRn antagonist. In some embodiments, the subject has an ESSDAI score of <5 and a clinESSDAI score of <5 following administration of the FcRn antagonist. In some embodiments, the ESSDAI score and/or the clinESSDAI score is measured 16 weeks and/or 24 weeks following administration of the FcRn antagonist. In some embodiments, the ESSDAI score and/or the clinESSDAI score is measured 16 weeks and/or 24 weeks following an initial administration of the FcRn antagonist.

In some embodiments, the subject shows an increase in Sjögren's Tool for Assessing Response (STAR) score following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the STAR score is measured 24 weeks following administration of the FcRn antagonist. In some embodiments, the STAR score is measured 24 weeks following an initial administration of the FcRn antagonist.

In some embodiments, the subject has a STAR score of ≥1, >2, ≥3, >4, ≥5, ≥6, ≥7, or >8 following administration of the FcRn antagonist. In some embodiments, the subject has a STAR score of >5 following administration of the FcRn antagonist. In some embodiments, the STAR score is measured 24 weeks following administration of the FcRn antagonist. In some embodiments, the STAR score is measured 24 weeks following an initial administration of an initial dose of the FcRn antagonist.

In some embodiments, the subject shows an improvement in total Multidimensional Fatigue Inventory (MFI) score, SF-36 physical component score, SF-36 mental component score,

PGA score, EQ-5D-5L score, VAS score, ESSPRI dryness score, ESSPRI fatigue score, ESSPRI pain score, and/or PASS score, following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the total MFI score, the SF-36 physical component score, the SF-36 mental component score, the PGA score, the EQ-5D-5L score, the VAS score, the ESSPRI dryness score, the ESSPRI fatigue score, the ESSPRI pain score, and/or the PASS score is measured at 16 weeks and/or 24 weeks following administration of the FcRn antagonist. In some embodiments, the total MFI score, the SF-36 physical component score, the SF-36 mental component score, the PGA score, the EQ-5D-5L score, the VAS score, the ESSPRI dryness score, the ESSPRI fatigue score, the ESSPRI pain score, and/or the PASS score is measured at 16 weeks and/or 24 weeks following an initial administration of the FcRn antagonist.

In some embodiments, the subject shows a decrease in MFI score following administration of the FcRn antagonist, compared to a baseline value. In some embodiments, the subject shows at least a 10% decrease in MFI score following administration of the FcRn antagonist, compared to a baseline value. In some embodiments, the subject shows at least a 20% decrease, at least a 30% decrease, at least a 40% decrease, or at least a 50% decrease in MFI score following administration of the FcRn antagonist, compared to a baseline value.

In some embodiments, the subject shows an increase in SF-36 physical component score following administration of the FcRn antagonist, compared to a baseline value. In some embodiments, the subject shows at least a 10% increase in SF-36 physical component score following administration of the FcRn antagonist, compared to a baseline value. In some embodiments, the subject shows at least a 20% increase, at least a 30% increase, at least a 40% increase, or at least a 50% increase in SF-36 physical component score following administration of the FcRn antagonist, compared to a baseline value.

In some embodiments, the subject shows an increase in SF-36 mental component score following administration of the FcRn antagonist, compared to a baseline value. In some embodiments, the subject shows at least a 10% increase in SF-36 mental component score following administration of the FcRn antagonist, compared to a baseline value. In some embodiments, the subject shows at least a 20% increase, at least a 30% increase, at least a 40% increase, or at least a 50% increase in SF-36 mental component score following administration of the FcRn antagonist, compared to a baseline value.

In some embodiments, the subject shows a decrease in PGA score following administration of the FcRn antagonist, compared to a baseline value. In some embodiments, the subject shows at least a 1, 2, 3, 4, 5, 6, 7, 8, or 9 point decrease in PGA score following administration of the FcRn antagonist, compared to a baseline value.

In some embodiments, the subject shows a decrease in EQ-5D-5L score in 1, 2, 3, 4, or 5 domains following administration of the FcRn antagonist, compared to a baseline value. In some embodiments, the subject shows a decrease of 1, 2, 3, or 4 points in EQ-5D-5L score in 1, 2, 3, 4, or 5 domains following administration of the FcRn antagonist, compared to a baseline value.

In some embodiments, the subject shows a decrease in VAS score following administration of the FcRn antagonist, compared to a baseline value.

In some embodiments, the subject shows a decrease in ESSPRI dryness score following administration of the FcRn antagonist, compared to a baseline value. In some embodiments, the subject shows at least a 1, 2, 3, 4, 5, 6, 7, 8, or 9 point decrease in ESSPRI dryness score following administration of the FcRn antagonist, compared to a baseline value.

In some embodiments, the subject shows a decrease in ESSPRI fatigue score following administration of the FcRn antagonist, compared to a baseline value. In some embodiments, the subject shows at least a 1, 2, 3, 4, 5, 6, 7, 8, or 9 point decrease in ESSPRI fatigue score following administration of the FcRn antagonist, compared to a baseline value.

In some embodiments, the subject shows a decrease in ESSPRI pain score following administration of the FcRn antagonist, compared to a baseline value. In some embodiments, the subject shows at least a 1, 2, 3, 4, 5, 6, 7, 8, or 9 point decrease in ESSPRI pain score following administration of the FcRn antagonist, compared to a baseline value.

In some embodiments, the subject shows an improvement in PASS score following administration of the FcRn antagonist, compared to a baseline value.

In some embodiments, the subject shows a change in SWSF rate, UWSF rate, Hocevar score, Schirmer's test score, and/or OSS, following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the SWSF rate, the UWSF rate, the Hocevar score, the Schirmer's test score, and/or the OSS is measured at 16 weeks and/or 24 weeks following administration of the FcRn antagonist. In some embodiments, the SWSF rate, the UWSF rate, the Hocevar score, the Schirmer's test score, and/or the OSS is measured at 16 weeks and/or 24 weeks following an initial administration of the FcRn antagonist.

In some embodiments, the subject shows an increase in SWSF rate following administration of the FcRn antagonist, compared to a baseline value. In some embodiments, the baseline value is ≤0.7, <0.6, or ≤0.5 mL/min. In some embodiments, the SWSF rate following administration of the FcRn antagonist is >0.7, >0.8, >0.9, >1.0, >1.1, >1.2, >1.3, >1.4, >1.5, >1.6, >1.7, >1.8, >1.9, or >2.0 mL/min. In some embodiments, the SWSF rate following administration of the FcRn antagonist is about 1.5-2.0 mL/min. In some embodiments, the subject shows at least a 10% increase, at least a 20% increase, at least a 30% increase, at least a 40% increase, or at least a 50% increase in SWSF rate following administration of the FcRn antagonist, compared to a baseline value.

In some embodiments, the subject shows an increase in UWSF rate following administration of the FcRn antagonist, compared to a baseline value. In some embodiments, the baseline value is ≤0.4, ≤0.3, ≤0.2, ≤0.1, or <0.05 mL/min. In some embodiments, the baseline value is 0 mL/min. In some embodiments, the baseline value is ≤0.1 mL/min. In some embodiments, the UWSF rate following administration of the FcRn antagonist is >0.1, >0.2, >0.3, or >0.4 mL/min. In some embodiments, the UWSF rate following administration of the FcRn antagonist is about 0.3-0.4 mL/min. In some embodiments, the subject shows at least a 5% increase, at least a 10% increase, at least a 20% increase, at least a 30% increase, at least a 40% increase, or at least a 50% increase in UWSF rate following administration of the FcRn antagonist, compared to a baseline value. In some embodiments, the subject shows at least a 25% increase in UWSF rate following administration of the FcRn antagonist, compared to a baseline value.

In some embodiments, the subject shows a decrease in Hocevar score following administration of the FcRn antagonist, compared to a baseline value. In some embodiments, the baseline value is ≥10, >15, ≥20, >25, >30, >35, >40, or >45. In some embodiments, the Hocevar score following administration of the FcRn antagonist is ≤25, ≤20, <15, <10, or ≤5. In some embodiments, the subject shows at least a 10% decrease, at least a 20% decrease, at least a 30% decrease, at least a 40% decrease, or at least a 50% decrease in Hocevar score following administration of the FcRn antagonist, compared to a baseline value. In some embodiments, the subject shows at least a 25% decrease in Hocevar score following administration of the FcRn antagonist, compared to a baseline value.

In some embodiments, the subject shows an increase in Schirmer's test score following administration of the FcRn antagonist, compared to a baseline value. In some embodiments, the baseline value is ≤5 mm/5 minutes on at least one eye. In some embodiments, the baseline value is ≤5 mm/5 minutes on both eyes. In some embodiments, the Schirmer's test score following administration of the FcRn antagonist is ≥5 mm/5 minutes, ≥10 mm/5 minutes, ≥15 mm/5 minutes on at least one eye. In some embodiments, the Schirmer's test score following administration of the FcRn antagonist is ≥5 mm/5 minutes, ≥10 mm/5 minutes, ≥15 mm/5 minutes on both eyes.

In some embodiments, the subject shows a decrease in OSS following administration of the FcRn antagonist, compared to a baseline value. In some embodiments, the baseline value is >5 points on at least one eye. In some embodiments, the baseline value is ≥3 points on at least one eye. In some embodiments, the baseline value is >5 points on both eyes. In some embodiments, the baseline value is ≥3 points on both eyes. In some embodiments, the subject shows at least a 1-point decrease, at least a 2-point decrease, at least a 3-point decrease, at least a 4-point decrease, at least a 5-point decrease, at least a 6-point decrease, at least a 7-point decrease, at least a 8-point decrease, at least a 9-point decrease, at least a 10-point decrease, at least a 11-point decrease, at least a 12-point decrease, or at least 13-point decrease in OSS on at least one eye following administration of the FcRn antagonist. In some embodiments, the subject shows at least a 1-point decrease, at least a 2-point decrease, at least a 3-point decrease, at least a 4-point decrease, at least a 5-point decrease, at least a 6-point decrease, at least a 7-point decrease, at least a 8-point decrease, at least a 9-point decrease, at least a 10-point decrease, at least a 11-point decrease, at least a 12-point decrease, or at least 13-point decrease in OSS on both eyes following administration of the FcRn antagonist. In some embodiments, the subject shows at least a 2-point decrease in OSS on at least one eye following administration of the FcRn antagonist. In some embodiments, the subject shows at least a 2-point decrease in OSS on both eyes following administration of the FcRn antagonist.

In some embodiments, after administering the FcRn antagonist to the subject, the subject exhibits a post-administration level of a serum autoantibody that is reduced as compared to a baseline level of the serum autoantibody obtained from the subject prior to administering the FcRn antagonist. In some embodiments, the post-administration level of the serum autoantibody is reduced by at least 10%, at least 20%, at least 30%, at least 40%, at least 50%, at least 60%, at least 70%, at least 80%, at least 90%, or 100%, as compared to the baseline level of the serum autoantibody obtained from the subject prior to administering the FcRn antagonist. In some embodiments, the post-administration level of the serum autoantibody is measured 1 week, 2 weeks, 4 weeks, 8 weeks, 12 weeks, 16 weeks, 20 weeks, and/or 24 weeks after administering the FcRn antagonist to the subject. In some embodiments, the post-administration level of the serum autoantibody is measured 1 week, 2 weeks, 4 weeks, 12 weeks, 16 weeks, 20 weeks, and/or 24 weeks after an initial administration of the FcRn antagonist to the subject. In some embodiments, the serum autoantibody is measured 4 weeks, 16 weeks, and/or 24 weeks following administration of the FcRn antagonist. In some embodiments, the serum autoantibody is measured 4 weeks, 16 weeks, and/or 24 weeks following an initial administration of the FcRn antagonist. Examples of autoantibodies associated with pSS include, but are not limited to, anti-Ro/SS-A antibodies, anti-La/SS-B antibodies, rheumatoid factor (RF), and antinuclear antibodies (ANA). In some embodiments, the autoantibody is an anti-Ro/SS-A antibody or an anti-La/SS-B antibody. In some embodiments, the autoantibody is an anti-Ro/SS-A antibody, an anti-La/SS-B antibody, RF, an antinuclear antibody or any combination thereof. In some embodiments, the autoantibody is an IgA-related autoantibody, an IgM-related autoantibody, or an IgG-related autoantibody.

In some embodiments, after administering the FcRn antagonist to the subject, the subject exhibits a post-administration level of a serum complement that is reduced as compared to a baseline level of the serum complement obtained from the subject prior to administering the FcRn antagonist. In some embodiments, the post-administration level of the serum complement is reduced by at least 10%, at least 20%, at least 30%, at least 40%, at least 50%, at least 60%, at least 70%, at least 80%, at least 90%, or 100%, as compared to the baseline level of the serum complement obtained from the subject prior to administering the FcRn antagonist. In some embodiments, the post-administration level of the serum complement is measured 4 weeks, 16 weeks, and/or 24 weeks after administering the FcRn antagonist to the subject. In some embodiments, the post-administration level of the serum complement is measured 4 weeks, 16 weeks, and/or 24 weeks after an initial administration of the FcRn antagonist to the subject. In some embodiments, the serum complement is selected from the group consisting of C3, C4, CH50, and C1q-binding circulating immune complexes.

In some embodiments, after administering the FcRn antagonist to the subject, the subject exhibits a post-administration level of circulating immune complexes that is reduced as compared to a baseline level of circulating immune complexes obtained from the subject prior to administering the FcRn antagonist. In some embodiments, the circulating immune complexes are selected from the group consisting of C3, C4, CH50, and C1q-binding circulating immune complexes. In some embodiments, the post-administration level of circulating immune complexes is reduced by at least 10%, at least 25%, at least 50%, at least 75%, at least 80%, at least 90%, at least 95%, at least 96%, at least 97%, at least 98%, or at least 99%, as compared to the baseline level of circulating immune complexes obtained from the subject prior to administering the FcRn antagonist. In some embodiments, the post-administration level of circulating immune complexes is measured 4 weeks, 16 weeks, and/or 24 weeks after administering the FcRn antagonist to the subject. In some embodiments, the post-administration level of circulating immune complexes is measured 4 weeks, 16 weeks, and/or 24 weeks after an initial administration of the FcRn antagonist to the subject.

In some embodiments, after administering the FcRn antagonist to the subject, the subject exhibits a post-administration level of serum IgG that is reduced as compared to a baseline level of serum IgG obtained from the subject prior to administering the FcRn antagonist. In some embodiments, the post-administration level of serum IgG is reduced by at least 10%, at least 20%, at least 30%, at least 40%, at least 50%, at least 60%, at least 70%, at least 80%, at least 90%, or 100%, as compared to the baseline level of serum IgG obtained from the subject prior to administering the FcRn antagonist. In some embodiments, the post-administration level of serum IgG is measured 1 week, 2 weeks, 4 weeks, 8 weeks, 12 weeks, 16 weeks, 20 weeks, and/or 24 weeks after administering the FcRn antagonist to the subject. In some embodiments, the post-administration level of serum IgG is measured 1 week, 2 weeks, 4 weeks, 8 weeks, 12 weeks, 16 weeks, 20 weeks, and/or 24 weeks after an initial administration of the FcRn antagonist to the subject. In some embodiments, the post-administration level of serum IgG is measured 4 weeks, 16 weeks, and/or 24 weeks after administering the FcRn antagonist to the subject. In some embodiments, the post-administration level of serum IgG is measured 4 weeks, 16 weeks, and/or 24 weeks after an initial administration of the FcRn antagonist to the subject.

In some embodiments, after administering the FcRn antagonist to the subject, the subject exhibits a post-administration level of cytokine or chemokine that is reduced as compared to a baseline level of cytokine or chemokine obtained from the subject prior to administering the FcRn antagonist. In some embodiments, the post-administration level of cytokine or chemokine is reduced by at least 10%, at least 20%, at least 30%, at least 40%, at least 50%, at least 60%, at least 70%, at least 80%, at least 90%, or 100%, as compared to the baseline level of cytokine or chemokine obtained from the subject prior to administering the FcRn antagonist. In some embodiments, the post-administration level of cytokine or chemokine is measured 4 weeks, 16 weeks, and/or 24 weeks after administering the FcRn antagonist to the subject. In some embodiments, the post-administration level of cytokine or chemokine is measured 4 weeks, 16 weeks, and/or 24 weeks after an initial administration of the FcRn antagonist to the subject. In some embodiments, the cytokine or chemokine is selected from the group consisting of B-cell activating factor (BAFF), type 1 IFN, IL 1B, IL 21, TNFα, IFNα, CD30, CD40 L, CCL5, CRP, and ferritin.

In some embodiments, after administering the FcRn antagonist to the subject, the level of albumin is not decreased in the subject following administration of the FcRn antagonist compared to a baseline level of albumin. In some embodiments, after administering the FcRn antagonist to the subject, the subject exhibits a post-administration level of albumin that is not decreased as compared to a baseline level of albumin obtained from the subject prior to administering the FcRn antagonist. In an embodiment, an albumin reduction of less than about 1%, 2%, 3%, 4%, or 5% compared to baseline albumin level is observed. In an embodiment, an albumin reduction of less than about 10% compared to baseline albumin level is observed. In some embodiments, the post-administration level of albumin is measured 4 weeks, 16 weeks, and/or 24 weeks after administering the FcRn antagonist to the subject. In some embodiments, the post-administration level of albumin is measured 4 weeks, 16 weeks, and/or 24 weeks after an initial administration of the FcRn antagonist to the subject.

In some embodiments, after administering the FcRn antagonist to the subject, the level of serum albumin is not reduced in the subject following administration of the FcRn antagonist compared to a baseline level of serum albumin. In some embodiments, after administering the FcRn antagonist to the subject, the subject exhibits a post-administration level of serum albumin that is not reduced as compared to a baseline level of serum albumin obtained from the subject prior to administering the FcRn antagonist. In an embodiment, a serum albumin reduction of less than about 1%, 2%, 3%, 4%, or 5% compared to baseline serum albumin level is observed. In an embodiment, a serum albumin reduction of less than about 10% compared to baseline serum albumin level is observed. In some embodiments, the post-administration level of serum albumin is measured 4 weeks, 12 weeks, and/or 24 weeks after administering the FcRn antagonist to the subject. In some embodiments, the post-administration level of serum albumin is measured 4 weeks, 12 weeks, and/or 24 weeks after an initial administration of the FcRn antagonist to the subject.

In some embodiments, the subject shows a change in saliva biomarker levels following administration of the FcRn antagonist, compared to a baseline value. In some embodiments, the subject shows a reduction in saliva biomarker levels following administration of the FcRn antagonist, compared to a baseline value. In some embodiments, the subject shows an increase in saliva biomarker levels following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the saliva biomarker levels are measured 4 weeks, 16 weeks, and/or 24 weeks following administration of the FcRn antagonist. In some embodiments, the saliva biomarker levels are measured 4 weeks, 16 weeks, and/or 24 weeks following an initial administration of the FcRn antagonist.

In some embodiments, the subject shows a change in salivary gland histology following administration of the FcRn antagonist, compared to a baseline value. In some embodiments, the salivary gland is one or more of a parotid gland, a submandibular gland, and a sublingual gland. In some embodiments, the baseline value is obtained from the subject prior to administration of the FcRn antagonist. In some embodiments, the baseline value is obtained from the subject prior to an initial administration of the FcRn antagonist. In some embodiments, the subject shows a change in immunophenotype, focus score, number of germinal centers, lymphoepithelial lesions, and/or gene expression per mm² in salivary gland parenchyma following administration of the FcRn antagonist, compared to a baseline value. In some embodiments, the change in the immunophenotype, the focus score, the number of germinal centers, the lymphoepithelial lesions, and/or the gene expression per mm² is measured 4 weeks and/or 24 weeks following administration of the FcRn antagonist. In some embodiments, the change in the immunophenotype, the focus score, the number of germinal centers, the lymphoepithelial lesions, and/or the gene expression per mm² is measured 4 weeks and/or 24 weeks following an initial administration of the FcRn antagonist.

Changes in salivary gland immunophenotype include, but are not limited to, changes in number of plasma cells (e.g., IgA, IgG, and/or IgM) per mm² parenchyma. In some embodiments, the subject shows a decrease in number of plasma cells in one or more salivary glands following administration of the FcRn antagonist, compared to a baseline value. In some embodiments, the subject shows a decrease in number of plasma cells of at least 5%, at least 10%, at least 15%, at least 20%, at least 25%, at least 30%, at least 40%, at least 50%, at least 60%, at least 70%, at least 80%, or at least 90%, compared to a baseline value. In some embodiments, the one or more salivary glands is a parotid gland.

In some embodiments, the subject shows a decrease in focus score following administration of the FcRn antagonist, compared to a baseline value. Focus score can be calculated by counting the number of inflammatory infiltrates of at least 50 cells present in 4 mm² of salivary gland area. In some embodiments, the baseline value is ≥1, >2, >3, >4, or ≥5. In some embodiments, the focus score following administration of the FcRn antagonist is <1, In some embodiments, the focus score following administration of the FcRn antagonist is 0. In some embodiments, the focus score is measured in a parotid gland.

In some embodiments, the subject shows a decrease in the number of germinal centers following administration of the FcRn antagonist, compared to a baseline value. A germinal center can be identified, for example, by H&E staining or CD21 staining, or a combination thereof. In some embodiments, the baseline value is the presence of one or more germinal centers in a salivary gland biopsy sample. In some embodiments, the number of germinal centers in a salivary gland biopsy sample following administration of the FcRn antagonist is <1 per mm². In some embodiments, the number of germinal centers in a salivary gland biopsy sample following administration of the FcRn antagonist is 0 per mm². In some embodiments, the salivary gland biopsy sample is a parotid gland biopsy sample.

In some embodiments, the subject shows a decrease in lymphoepithelial lesions following administration of the FcRn antagonist, compared to a baseline value. Lymphoepithelial lesions are characteristic manifestations of typical duct lesions in salivary gland(s) of subjects with SS, resulting from infiltration of lymphocytes into the hyperplasia of basal cells in the duct. Presence of lymphoepithelial lesions in parotid gland biopsy samples often indicates risk for occurrence of lymphoma. In some embodiments, the baseline value is the presence of one or more lymphoepithelial lesions in a salivary gland biopsy sample. In some embodiments, the number of lymphoepithelial lesions in a salivary gland biopsy sample following administration of the FcRn antagonist is <1 per mm². In some embodiments, the number of lymphoepithelial lesions in a salivary gland biopsy sample following administration of the FcRn antagonist is 0 per mm². In some embodiments, the salivary gland biopsy sample is a parotid gland biopsy sample.

In some embodiments, the subject shows a change in salivary gland gene expression profile following administration of the FcRn antagonist, compared to a baseline value. In some embodiments, the change in salivary gland gene expression profile is measured using RNA sequencing. In some embodiments, the subject shows a change in expression of genes relevant to participant immune status and/or related pathways, including, but not limited to, the IFN pathway. In some embodiments, the salivary gland is a parotid gland.

In some embodiments, the subject shows a change in blood biomarkers following administration of the FcRn antagonist, compared to a baseline value. In some embodiments, the baseline value is obtained from the subject prior to administration of the FcRn antagonist. In some embodiments, the baseline value is obtained from the subject prior to an initial administration of the FcRn antagonist. In some embodiments, the change in blood biomarkers is detected by immunophenotyping and/or RNA sequencing. In some embodiments, the subject shows a change in gene expression profile in blood biomarkers following administration of the FcRn antagonist, compared to a baseline value. In some embodiments, the change in gene expression profile in blood biomarkers is measured using RNA sequencing. In some embodiments, the subject shows a change in expression of genes relevant to participant immune status and/or related pathways, including, but not limited to, the IFN pathway. In some embodiments, the gene expression profile is measured at 4 weeks, 16 weeks, and/or 24 weeks following administration of the FcRn antagonist. In some embodiments, the gene expression profile is measured at 4 weeks, 16 weeks, and/or 24 weeks following an initial administration of the FcRn antagonist.

In some embodiments, the change in immunophenotyping in peripheral blood is measured using flow cytometry. In some embodiments, peripheral blood mononuclear cells (PBMCs) are used for immunophenotyping using flow cytometry, including but not limited to the assessment of the proportion of B cells within the CD45+ population. In some embodiments, the immunophenotype is measured at 4 weeks and/or 24 weeks following administration of the FcRn antagonist. In some embodiments, the immunophenotype is measured at 4 weeks and/or 24 weeks following an initial administration of the FcRn antagonist.

In an embodiment, the subject is any human or non-human animal. In an embodiment, the subject is a human or non-human mammal. In an embodiment, the subject is a human.

### Examples

The following examples are offered by way of illustration, and not by way of limitation.

### Example 1: Investigation of Efficacy and Safety of Efgartigimod in Adult Patients with Primary Sjögren's Syndrome (pSS)

Primary Sjögren's syndrome (pSS) is a chronic, progressive autoimmune disease of unclear etiology, typically presented as an exocrinopathy. Along with symptoms of extensive dryness, manifestations include profound fatigue, chronic pain, extraglandular organ system involvement, and increased risk of lymphomas. pSS is characterized by mononuclear inflammatory infiltrates and IgG plasma cells in salivary and lacrimal glands that lead to irreversible destruction of the glandular tissue and is accompanied by sensation of dryness of mouth and eyes. B cells play a central role in the immunopathogenesis and exhibit signs of hyperactivity. In addition, autoantibodies can create immune complexes that maintain and amplify the production of IFN alpha. This combination results in a cycle of immune activation that leads to tissue damage. A hallmark of pSS is B cell hyperactivity, causing a vicious cycle of immune activation through cytokine production, antigen presentation, and autoantibody secretion, potentially causing tissue damage. Currently, no immunomodulatory treatment is available for pSS.

For many decades, the clinical needs for pSS have been left unresolved because of the rareness of the disease and the complexity of the underlying pathogenic mechanisms. It has become clear that B cell activation and development of autoantibodies play an important role in pSS.

This study aims to evaluate the effect of efgartigimod, an FcRn antagonist that can rapidly reduce IgG, including pathogenic antibodies. Efgartigimod has the potential to successfully treat pSS and improve disease manifestations by the reduction of IgG autoantibodies in pSS.

### A. Study Design

### Overall Design

This is a randomized, double-blinded, placebo-controlled, phase 2 study.

For participants not enrolling in the open-label extension (OLE) study, the total study duration is approximately 36 weeks comprising:
Screening period of ≤4 weeks
Treatment period of 24 weeks
Follow-up period of 56 days.

For participants enrolling in the open-label extension (OLE) study, the total study duration is approximately 28 weeks comprising:
Screening period of ≤4 weeks
Treatment period of 24 weeks.

The study population is adult patients with pSS, per American College of Rheumatology/European Alliance of Associations for Rheumatology (ACR/EULAR) 2016 classification criteria, with at least a moderate level of systemic disease activity (EULAR Sjögren's Syndrome Disease Activity Index (ESSDAI) ≥5). Participants are randomized to receive efgartigimod 10 mg/kg or placebo in a 2:1 ratio, respectively. All participants receive efgartigimod IV 10 mg/kg or placebo once weekly for 24 weeks during the treatment period. IMP (efgartigimod or placebo) is administered in an approximately 1-hour IV infusion by site staff or a home nurse. The final dose is administered at week 23. At week 24, eligible participants may roll over into a single-arm OLE.

### Selection of Primary and Secondary Endpoints

This study aims to evaluate the efficacy and safety of efgartigimod compared to placebo in participants with pSS. The study design is randomized, double-blinded, and placebo-controlled to evaluate the effect of efgartigimod administered as an IV infusion compared to placebo. The study consists of a treatment period where all participants receive weekly IV infusions for 24 weeks. As there are no standardized approved therapies for pSS, the comparison to placebo is justified.

The primary endpoint is the effect of efgartigimod IV compared to placebo on CRESS (Composite of Relevant Endpoints for Sjögren's Syndrome; described further below). The CRESS composite measures systemic disease activity, patient-reported symptoms, tear gland function, salivary gland function, and serology, developed to assess treatment efficacy in participants with pSS. CRESS was selected as the primary efficacy endpoint because a composite is presumed to be more appropriate in demonstrating drug efficacy compared to an endpoint that reports on a single aspect of this heterogeneous disease. In randomized controlled studies that previously showed negative primary endpoint results using ESSDAI, post hoc analysis of the study data using CRESS resulted in higher response rates in participants. Use of CRESS also resulted in decreased placebo response rates compared with the use of the ESSDAI minimal clinically important improvement, which is essential to demonstrating treatment efficacy.

The secondary endpoints complement the primary endpoint and provide additional information on efficacy (ESSDAI, clinical ESSDAI [clinESSDAI], EULAR Sjogren's syndrome Patient Reported Index [ESSPRI], Sjogren's Tool for Assessing Response [STAR] [all described further below]), histology, safety, PK, PD, and immunogenicity.

### End of Study

The end of study is defined as the date of the last participant's last visit. A participant has completed the study if the treatment period (or follow-up period, if applicable) has been completed.
Participants rolling over to the OLE study complete this study at week 24.
Participants not rolling over to the OLE study complete this study after the safety follow-up visit (SFV) or early discontinuation visit (EDV), as applicable. If a participant continued in the study after discontinuing IMP, this is week 24 or at the SFV (if permanent IMP discontinuation is <56 days from week 24).

### B. Study Population

Prospective approval of protocol deviations to recruitment and enrollment criteria, also known as protocol waivers or exemptions, is not permitted.

Inclusion criteria

Participants are eligible for study inclusion only if all of the following criteria apply:
Reached age of consent when signing the informed consent form;
Capable of providing signed informed consent, which includes compliance with the requirements and restrictions listed in the informed consent form and this protocol;
Meets the following criteria:
   ACR/EULAR 2016 pSS who met criteria≤7 years before screening (see below);
   ESSDAI≥5;
   Ro/SS-A positive;
   Residual salivary flow (unstimulated whole salivary flow (UWSF) rate>0 and/or stimulated whole salivary flow (SWSF) rate>0.10);
Agree to use contraceptives consistent with local regulations regarding the methods of contraception for those participating in clinical studies and the following:
   Female participants of childbearing potential must have a negative serum pregnancy test at screening and a negative urine pregnancy test at baseline before receiving IMP.

The ACR/EULAR classification of pSS applies to any individual who meets the following inclusion and exclusion criteria and has a score of >4 when summing the weights from the items presented in Table S1.

ACR/EULAR pSS classification inclusion criteria apply to any patient with ≥1 symptom of ocular or oral dryness, defined as a positive response to >1 of the following questions:
Have you had daily, persistent, troublesome dry eyes for more than 3 months?
Do you have a recurrent sensation of sand or gravel in the eyes?
Do you use tear substitutes more than 3 times a day?
Have you had a daily feeling of dry mouth for more than 3 months?

Do you frequently drink liquids to aid in swallowing dry food?

OR suspicion of pSS from the ESSDAI questionnaire (with >1 positive domain item).

Prior diagnosis of any of the following conditions would exclude diagnosis of pSS according to ACR/EULAR criteria and participation in pSS studies or therapeutic studies because of overlapping clinical features or interference with criteria tests:
AIDS;
Active hepatitis C infection (with positive PCR);
Sarcoidosis;
Amyloidosis;
Graft-versus-host disease;
History of head and neck radiation treatment;
IgG4-related disease.

TABLE S1
ACR/EULAR Classification Criteria for
Primary Sjögren's Syndrome

| | Weight/ |
|---|---|
| Item | Score |
| Labial salivary gland with focal lymphocytic sialadenitis and | 3 |
| focus score of ≥1^{a} | |
| Anti-Ro/SS-A positive | 3 |
| OSS of ≥5 (or van Bijsterfeld score of ≥4) on at least 1 eye | 1 |
| Schirmer's test of ≤5 mm/5 minutes on at least 1 eye^{b} | 1 |
| UWSF rate of ≤0.1 mL/min^{b} | 1 |

ACR = American College of Rheumatology;
EULAR = European Alliance of Associations for Rheumatology;
OSS = ocular staining score;
SS-A = Sjögren's syndrome-related antigen A;
UWSF = unstimulated whole salivary flow
^{a}A pathologist with expertise in the diagnosis of focal lymphocytic sialadenitis and focus score count (based on number of foci per 4 mm²) should perform the histopathologic examination following a protocol by Daniels et al. Arthritis Rheumatol. 2011; 63(7): 2021-2030.
^{b}Patients who are normally taking anticholinergic drugs should be evaluated for objective signs of salivary hypofunction and ocular dryness after a sufficient interval off these medications so that these components can be a valid measure of oral and ocular dryness.

### Exclusion Criteria

Participants are excluded from the study if any of the following criteria apply:
Known autoimmune disease or any medical condition that, in the investigator's judgment, would interfere with an accurate assessment of clinical symptoms of pSS or puts the participant at undue risk;
History of malignancy unless considered cured by adequate treatment with no evidence of recurrence for >3 years before the first administration of IMP. Adequately treated participants with the following cancers may be included at any time:
   Basal cell or squamous cell skin cancer;
   Carcinoma in situ of the cervix;
   Carcinoma in situ of the breast;
   Incidental histological finding of prostate cancer (TNM stage T1a or T1b);
Clinically significant uncontrolled active or chronic bacterial, viral, or fungal infection;
Positive serum test at screening for an active infection with any of the following:
   Hepatitis B virus (HBV) that is indicative of an acute or chronic infection, unless associated with a negative HB surface antigen (HBsAg) or negative HBV DNA test;
   Hepatitis C virus (HCV) based on HCV antibody assay unless a negative RNA test is available;
   Human immunodeficiency virus (HIV) based on test results of a CD4 count of <200 cells/mm³ that are associated with an AIDS-defining condition;
   HIV based on test results of a CD4 count of >200 cells/mm³ not adequately treated with antiviral therapy;
Clinically significant disease, recent major surgery (within 3 months of screening), or intends to have surgery during the study; or any other condition that in the opinion of the investigator could confound the results of the study or put the participant at undue risk;
Current participation in another interventional clinical study;
Known hypersensitivity to IMP or 1 of its excipients;
History (within 12 months of screening) of or current alcohol, drug, or medication abuse;
Pregnant or lactating or intention to become pregnant during the study;
Previous participation in an efgartigimod clinical study and treatment with ≥1 dose of IMP;
Total IgG <4 g/L at screening;
Secondary Sjögren's syndrome overlap syndromes where another confirmed autoimmune rheumatic or systemic inflammatory condition (e.g., rheumatoid arthritis, systemic lupus erythematosus, scleroderma, inflammatory bowel disease) is the primary diagnosis;
Positive SARS-COV-2 PCR test at screening;
Any severe systemic pSS manifestation that may put the participant at undue risk based on the investigator's opinion;
Intravenous immunoglobulin (IVIg), subcutaneous immunoglobulin (SCIg), or plasmapheresis/plasma exchange (PLEX)<12 weeks before screening or during screening;
Live or live-attenuated vaccine <4 weeks before screening or during screening;
Pilocarpine and/or any other pharmacological stimulant for salivary and lacrimal glands≤4 weeks from screening or during screening;
Systemic corticosteroids≤4 weeks from screening or during screening;
Antimalarials, conventional disease-modifying antirheumatic drugs (DMARDs), or Janus kinase (JAK) inhibitors <4 weeks from screening or during screening;
Biologic DMARDs≤24 weeks from screening or during screening;
Nonbiologic IMP in another clinical study <12 weeks or 5 half-lives (whichever is longer) or a biologic IMP <24 weeks or 5 half-lives (whichever is longer) before screening or during screening;
Chinese traditional medicine with known immunomodulatory action.

### C. IMP(s) and Concomitant Therapy

Investigational medicinal product (IMP) is defined as any investigational intervention(s), marketed product(s), placebo, or medical device(s) intended to be administered to a study participant according to the study protocol.

### IMP(s) Administered

The IMP in this study includes efgartigimod IV and matching placebo (with the same formulation but without the active ingredient of efgartigimod), as described in Table S2:

TABLE S2
IMP(s) administered

| | | |
|---|---|---|
| Intervention Label | Efgartigimod IV | Placebo |
| Intervention Name | Efgartigimod IV | Placebo |
| Intervention Description | Sterile, colorless, clear | Sterile, colorless, clear |
| | concentrate solution for IV | concentrate solution for infusion, |
| | infusion efgartigimod 20 mg/mL, | with the same excipients as |
| | administered IV | efgartigimod IV, but without the |
| | | active ingredient (efgartigimod) |
| Type | Biologic | Placebo |
| Dose Formulation | Concentrate for solution for IV | Concentrate for solution for IV |
| | infusion | infusion |
| Unit Dose Strength(s) | 20 mg/mL | Not applicable |
| Dosage Level(s) | 10 mg/kg weekly x24 weeks | Not applicable |
| Route of Administration | IV infusion | IV infusion |
| Use | Experimental | Placebo-comparator |
| Sourcing | Provided centrally by the sponsor | Provided centrally by the sponsor |
| Packaging and Labeling | IMP provided in glass | IMP provided in glass |
| | vials. Each vial labeled as | vials. Each vial labeled as |
| | required per country requirements | required per country requirements |
| Former Name | ARGX-113 | Not applicable |

The 10 mg/kg efgartigimod dose is based on body weight, and the maximum total dose per efgartigimod infusion is 1200 mg for participants who weigh ≥120 kg. The dose level is recalculated for body weight changes of ±10%.

Any medication or vaccine (including over-the-counter or prescription medicines, vitamins, and/or herbal supplements [including Chinese traditional medicine]) or other specific categories of interest that the participant is receiving at the time of screening or receives during the study participation must be recorded and include the following information: reason for use, dates of administration (including start and end dates), and dosage information (e.g., dose and frequency).

### Prohibited Medications

The following medications/treatments are prohibited while the participant receives IMP:
SCIg or IVIg;
PLEX;
Live or live-attenuated vaccines;
Pilocarpine and/or any other pharmacological stimulant for salivary and lacrimal glands;
Systemic corticosteroids;
Antimalarials, conventional DMARDs, JAK inhibitors;
Biologic DMARDs (e.g., rituximab, other monoclonal antibodies);
Pharmacological topical ophthalmic agents (e.g., NSAIDs, corticosteroids, cyclosporine);
IMP in another clinical study;
Chinese traditional medicine with known immunomodulatory action.
Anticholinergic agents are permitted if the dose is stable during the screening and treatment periods.
Topical symptomatic medications for pSS are permitted but restrictions apply:
At the baseline, week 16, week 24, and EDV/IMP discontinuation visits, participants are required to withhold ophthalmic lubricants, ophthalmic lubricating ointments, hydroxyl cellulose ophthalmic inserts, and saliva substitutes before efficacy assessments are completed.

### D. Study Assessments and Procedures

TABLE S3
Schedule of activities

| | | Study week | | | | | | | | | | | | | | |
|---|---|---|---|---|---|---|---|---|---|---|---|---|---|---|---|---|
| Study day | SCR | | 1 | 2 | 3 | 4 | 5 | 6 | 7 | 8 | 9 | 10 | 11 | 12 | 13 | 14 |
| ±Days | −28 | BL | ±1 | ±1 | ±1 | ±1 | ±1 | ±1 | ±1 | ±1 | ±1 | ±1 | ±1 | ±1 | ±1 | ±1 |
| Visits^{d, e} | to −2 | 1 | 2 | 3 | 4 | 5 | 6 | 7 | 8 | 9 | 10 | 11 | 12 | 13 | 14 | 15 |
| Informed | X | | | | | | | | | | | | | | | |
| consent | | | | | | | | | | | | | | | | |
| Inclusion and | X | X | | | | | | | | | | | | | | |
| exclusion | | | | | | | | | | | | | | | | |
| criteria | | | | | | | | | | | | | | | | |
| Medical/ | X | | | | | | | | | | | | | | | |
| surgical history^{f} | | | | | | | | | | | | | | | | |
| Demography^{g} | X | | | | | | | | | | | | | | | |
| Physical | X | X | | | | | | | | | | | | | | |
| examination^{h} | | | | | | | | | | | | | | | | |
| Brief physical | | | X | X | | X | | | | X | | | | X | | |
| examination | | | | | | | | | | | | | | | | |
| (symptom | | | | | | | | | | | | | | | | |
| driven)ⁱ | | | | | | | | | | | | | | | | |
| Vital signs^{j} | X | X | X | X | X | X | X | X | X | X | X | X | X | X | X | X |
| ECG | X | X | | | | | | | | | | | | | | |
| Randomization | | X | | | | | | | | | | | | | | |

Glandular function

| | | |
|---|---|---|
| UWSF/SWSF | X | X |
| Schirmer's test | | X |
| OSS | | X |
| SGUS | | X |

Histology

| | |
|---|---|
| Parotid biopsy | X |
| (selected sites | |
| only)^{k} | |

Safety laboratory assessments

| | | | | | |
|---|---|---|---|---|---|
| Clinical | X | X | X | X | X |
| laboratory tests | | | | | |
| (hematology and | | | | | |
| chemistry)^{l} | | | | | |
| Serology | X | | | | |
| HIV/hepatitis^{l} | | | | | |
| Urinalysis^{l} | X | X | | | |
| Pregnancy | X | X | X | X | X |
| testing^{l, m} | | | | | |
| PCR COVID-19 | X | | | | |
| test^{l, n} | | | | | |

Blood samples

| | | | | | | | |
|---|---|---|---|---|---|---|---|
| Exploratory | | X | | | X | | |
| biomarkers^{o, p} | | | | | | | |
| PK^{r} | | X | X | X | X | X | X |
| Total IgG^{l, o} | X | X | X | X | X | X | X |
| RF^{o} | | X | X | X | X | X | X |
| Anti-Ro/SS-A, | X | X | X | X | X | X | X |
| anti-La/SS-B | | | | | | | |
| autoanti- | | | | | | | |
| bodies^{l, o} | | | | | | | |
| Immuno- | X | X | | X | X | X | X |
| genicity^{l, o} | | | | | | | |

Saliva samples

| | | |
|---|---|---|
| Exploratory | X | X |
| biomarkers^{s} | | |

Systemic disease activity

| | | |
|---|---|---|
| ESSDAI^{t} | X | X |
| clinESSDAI | | X |

Patient-reported outcome questionnaires^{u}

| | | | | | | | | | | | | | | | |
|---|---|---|---|---|---|---|---|---|---|---|---|---|---|---|---|
| ESSPRI | X | | | | | | | | | | | | | | |
| MFI | X | | | | | | | | | | | | | | |
| PGA | X | | | | | | | | | | | | | | |
| SF-36 | X | | | | | | | | | | | | | | |
| EQ-5D-5L | X | | | | | | | | | | | | | | |
| PASS | X | | | | | | | | | | | | | | |
| IMP | X | X | X | X | X | X | X | X | X | X | X | X | X | X | X |
| administration^{v} | | | | | | | | | | | | | | | |

| | |
|---|---|
| Concomitant | Continuous monitoring |
| therapies^{w} | |
| AEs^{w} | Continuous monitoring |

| | Study week | | | | | | | | | | | | |
|---|---|---|---|---|---|---|---|---|---|---|---|---|---|
| | | | | | | | | | | | | EDV^{c} | SFV^{a} |
| | | | | | | | | | | | | (≤7 d | (56 d |
| | | | | | | | | | | | | after | after |
| | | | | | | | | | | | IMP | final | final |
| Study day | 15 | 16 | 17 | 18 | 19 | 20 | 21 | 22 | 23 | 24 | d/c | dose) | dose) |
| ±Days | ±1 | ±1 | ±1 | ±1 | ±1 | ±1 | ±1 | ±1 | ±1 | ±1 | visit^{b} | +2 | ±3 |
| Visits^{d, e} | 16 | 17 | 18 | 19 | 20 | 21 | 22 | 23 | 24 | 25 | NA | NA | NA |
| Informed | | | | | | | | | | | | | |
| consent | | | | | | | | | | | | | |
| Inclusion and | | | | | | | | | | | | | |
| exclusion | | | | | | | | | | | | | |
| criteria | | | | | | | | | | | | | |
| Medical/ | | | | | | | | | | | | | |
| surgical history^{f} | | | | | | | | | | | | | |
| Demography^{g} | | | | | | | | | | | | | |
| Physical | | | | | | | | | | | | | |
| examination^{h} | | | | | | | | | | | | | |
| Brief physical | X | | | | | X | | | | X | X | X | X |
| examination | | | | | | | | | | | | | |
| (symptom | | | | | | | | | | | | | |
| driven)ⁱ | | | | | | | | | | | | | |
| Vital signs^{j} | X | X | X | X | X | X | X | X | X | X | X | X | X |
| ECG | X | | | | | | | | | X | X | X | X |
| Randomization | | | | | | | | | | | | | |
| Glandular function | | | | | | | | | | | | | |
| UWSF/SWSF | X | | | | | | | | | X | X | X | |
| Schirmer's test | X | | | | | | | | | X | X | X | |
| OSS | X | | | | | | | | | X | X | X | |
| SGUS | X | | | | | | | | | X | X | X | |
| Histology | | | | | | | | | | | | | |
| Parotid biopsy | | | | | | | | | | X | X | X | |
| (selected sites | | | | | | | | | | | | | |
| only)^{k} | | | | | | | | | | | | | |
| Safety laboratory assessments | | | | | | | | | | | | | |
| Clinical | X | | | | | X | | | | X | X | X | X |
| laboratory tests | | | | | | | | | | | | | |
| (hematology and | | | | | | | | | | | | | |
| chemistry)^{l} | | | | | | | | | | | | | |
| Serology | | | | | | | | | | | | | |
| HIV/hepatitis^{l} | | | | | | | | | | | | | |
| Urinalysis^{l} | X | | | | | | | | | X | X | X | X |
| Pregnancy | X | | | | | X | | | | X | X | X | X |
| testing^{l, m} | | | | | | | | | | | | | |
| PCR COVID-19 | | | | | | | | | | | | | |
| test^{l, n} | | | | | | | | | | | | | |
| Blood samples | | | | | | | | | | | | | |
| Exploratory | X^{q} | | | | | | | | | X | X | X | |
| biomarkers^{o, p} | | | | | | | | | | | | | |
| PK^{r} | X | | | | | X | | | | X | X | X | X |
| Total IgG^{l, o} | X | | | | | X | | | | X | X | X | X |
| RF^{o} | X | | | | | X | | | | X | X | X | |
| Anti-Ro/SS-A, | X | | | | | X | | | | X | X | X | X |
| anti-La/SS-B | | | | | | | | | | | | | |
| autoanti- | | | | | | | | | | | | | |
| bodies^{l, o} | | | | | | | | | | | | | |
| Immuno- | X | | | | | X | | | | X | X | X | X |
| genicity^{l, o} | | | | | | | | | | | | | |
| Saliva samples | | | | | | | | | | | | | |
| Exploratory | X | | | | | | | | | X | X | X | |
| biomarkers^{s} | | | | | | | | | | | | | |
| Systemic disease activity | | | | | | | | | | | | | |
| ESSDAI^{t} | X | | | | | | | | | X | X | X | |
| clinESSDAI | X | | | | | | | | | X | X | X | |
| Patient-reported outcome questionnaires^{u} | | | | | | | | | | | | | |
| ESSPRI | X | | | | | | | | | X | X | X | |
| MFI | X | | | | | | | | | X | X | X | |
| PGA | X | | | | | | | | | X | X | X | |
| SF-36 | X | | | | | | | | | X | X | X | |
| EQ-5D-5L | X | | | | | | | | | X | X | X | |
| PASS | X | | | | | | | | | X | X | X | |
| IMP | X | X | X | X | X | X | X | X | X | | | | |
| administration^{v} | | | | | | | | | | | | | |

| | |
|---|---|
| Concomitant | Continuous monitoring |
| therapies^{w} | |
| AEs^{w} | Continuous monitoring |

BL = baseline;
clinESSDAI = clinical ESSDAI;
D = day;
d/c = discontinuation;
ECG = electrocardiogram;
EDV = early discontinuation visit;
ESSDAI = EULAR Sjögren's syndrome disease activity index;
ESSPRI = EULAR Sjögren's Syndrome Patient Reported Index;
EULAR = European Alliance of Associations for Rheumatology;
ICF = informed consent form;
IgG = immunoglobulin G;
IMP = investigational medicinal product;
IV = intravenous;
MFI = Multidimensional Fatigue Inventory;
NA = not applicable;
OSS = ocular staining score;
PASS = patient acceptable symptom state;
PCR = polymerase chain reaction;
PD = pharmacodynamics;
PGA = patient global assessment;
PK = pharmacokinetics;
RF = rheumatoid factor;
SCR = screening;
SFV = safety follow-up visit;
SF-36 = 36-Item Short Form Survey;
SGUS = salivary gland ultrasonography;
SS-A = Sjögren's syndrome-related antigen A;
SS-B = Sjögren's syndrome-related antigen B;
SWSF = stimulated whole salivary flow;
UWSF = unstimulated whole salivary flow;
W = week
^{a}For participants who discontinue IMP but remain in the study attending on-site visits, safety follow-up assessments occur on the previously scheduled visit closest to 56 days (±3 days) from the final IMP dose.
^{b}The IMP discontinuation visit is performed at the next scheduled visit after permanent IMP discontinuation and applies for participants who discontinue IMP but remain in the study. Participants who permanently discontinue IMP perform the IMP discontinuation visit and then are asked to proceed with their regularly scheduled visits.
^{c}The EDV applies for participants who discontinue the study.
^{d}Visits 4, 6-8, 10-12, 14-16, 18-20, and 22-24 may be performed in the participant's home by a home nurse.
^{e}Safety, efficacy, and predose sampling activities are performed before administering IMP.
^{f}Medical/surgical history includes all significant findings, surgeries, and preexisting conditions (including allergies, if any) present at screening including start and end date, if known.
^{g}Demographic characteristics comprise age, birth year, sex, race, and ethnicity (per local regulations). Race and ethnicity data are source verified only if permitted by local laws.
^{h}Physical examination includes height and weight. Height is measured at the baseline visit only.
ⁱThe brief physical examination is symptom driven and is performed as necessary to assess the ESSDAI. It also includes weight.
^{j}Vital signs are all measured before collecting any blood sample or administering IMP infusions.
^{k}Parotid biopsy is collected for immunophenotyping, assessment of germinal centers, lymphoepithelial lesions, and gene expression profiling. Refer to additional information about gene expression profiling below.
^{l}Screening laboratory tests is performed at a central laboratory.
^{m}Pregnancy testing uses a highly sensitive serum test at screening, and a urine test at all subsequent visits before IMP administration. Local regulations are followed if more stringent or frequent testing is required.
ⁿCOVID-19 testing occurs within 72 hours of baseline. Participants are tested for SARS-CoV-2 if they are symptomatic or if applicable law requires testing. COVID-19 testing is performed at a central or local laboratory.
^{o}Blood samples are taken predose preferably within 2 hours before administering IMP.
^{p}Blood samples are collected for immunophenotyping, cytokine and chemokine profiles, C1q immune complexes, autoantibodies, complement activation, and gene expression profiling.
^{q}Exploratory biomarkers that are measured by flow cytometry are not assessed at week 16.
^{r}Blood for PK analyses is collected predose (preferably within 2 hours before the infusion) and postdose (within 30 minutes after the end of the infusion).
^{s}Salivary samples are collected for evaluation of salivary proteins. Refer to additional information about autoantibody analysis below.
^{t}The biological domain is blinded postbaseline and sites score only the clinical domains of the ESSDAI.
^{u}Patient-reported outcome questionnaires are administered before any other study visit procedure where safe, and before discussions with staff about disease/treatment.
^{v}The IMP is administered as an approximately 1-hour IV infusion. Participants are monitored for safety for ≥30 minutes after the end of IMP administration.
^{w}AEs and use of concomitant therapies are continuously monitored from the time the ICF is signed until the last study-related activity.

### Cress

The primary efficacy endpoint is the proportion of responders on ≥3 of 5 items at week 24 using CRESS. CRESS was developed to assess treatment efficacy in participants with pSS. CRESS consists of the following items, with definitions of treatment response and lower disease activity:
Systemic disease activity: as measured with clinESSDAI (see below)
   Response is defined as a score of <5 points.
Patient-reported symptoms: as measured with ESSPRI (see below)
   Response is defined as a decrease of ≥1 point or >15% from baseline.
Tear gland function: as measured with Schirmer's test (see below) and OSS (see below)
   If Schirmer's test is ≤5 mm at baseline (abnormal), a response is defined as an increase of at least 5 mm from baseline. OR
   If OSS is ≥3 points at baseline (abnormal), a response is defined as a decrease of at least 2 points from baseline. OR
   If both OSS and Schirmer's scores are normal at baseline, a response is defined as no change that results in an abnormal OSS or Schirmer's score.
Salivary gland function: as measured with UWSF (see below) and salivary gland ultrasonography (SGUS)
   UWSF: at least 25% increase in score, or if score is 0 mL/min at baseline, any increase from baseline. OR
   SGUS: at least 25% decrease in total Hocevar score from baseline.
Serology: as measured with serum IgG and RF
   Rheumatoid factor (RF): decrease of at least 25% from baseline. OR
   IgG: reduction of at least 10% from baseline.

### Parotid Gland Histology

A secondary efficacy measure is the relative amount of lymphocytic infiltrate that can be assessed with CD45 immunohistochemical staining of the parotid gland. Increased lymphocytic infiltrate positive for CD45 has been observed in parotid gland biopsies of participants with pSS. Additionally, the ratio of B/B+T cells are assessed as a secondary outcome measure.

Furthermore, the following are assessed in the parotid gland as exploratory measures:
Changes in immunophenotype, including but not limited to plasma cells (IgA, IgG and IgM) per mm² parenchyma;
Focus score;
Number of germinal centers and lymphoepithelial lesions per mm² in parotid gland parenchyma;
Gene expression:
   Gene expression analysis may be conducted using RNA sequencing for genes relevant to the participant immune status and related pathways, including the IFN pathway, to understand the response to efgartigimod therapy. Testing is optional if it is categorized as genetic testing by local regulations.

### ESSDAI

The ESSDAI was designed to measure disease activity in patients with pSS. The ESSDAI consists of 12 domains, 11 related to organ involvement (cutaneous, pulmonary, renal, articular, muscular, peripheral nervous system, central nervous system, hematological, glandular, constitutional, and lymphadenopathic) and 1 biological domain reflecting B-cell activity. The activity levels of each domain (range: 0-3 points) are multiplied by their respective weights (range: 1-6 points) to obtain the total score (Table S4).

TABLE S4
ESSDAI: Domain and Item Definitions and Weights

| Domain [Weight] | Activity level | Description |
|---|---|---|
| Constitutional [3] | No = 0 | Absence of the following symptoms |
| Exclusion of fever of | Low = 1 | Mild or intermittent fever (37.5-38.5° C.)/night sweats and/or |
| infectious origin and | | involuntary weight loss of 5 to 10% of body weight |
| voluntary weight loss | Moderate = 2 | Severe fever (>38.5° C.)/night sweats and/or involuntary |
| | | weight loss of >10% of body weight |
| Lymphadenopathy [4] | No = 0 | Absence of the following features |
| Exclusion of infection | Low = 1 | Lymphadenopathy ≥1 cm in any nodal region or ≥2 cm in |
| | | inguinal region |
| | Moderate = 2 | Lymphadenopathy ≥2 cm in any nodal region or ≥3 cm in |
| | | inguinal region, and/or splenomegaly (clinically palpable or |
| | | assessed by imaging) |
| | High = 3 | Current malignant B-cell proliferative disorder |
| Glandular [2] | No = 0 | Absence of glandular swelling |
| Exclusion of stone or | Low = 1 | Small glandular swelling with enlarged parotid (≤3 cm), or |
| infection | | limited submandibular or lachrymal swelling |
| | Moderate = 2 | Major glandular swelling with enlarged parotid (>3 cm), or |
| | | important submandibular or lachrymal swelling |
| Articular [2] | No = 0 | Absence of currently active articular involvement |
| Exclusion of osteoarthritis | Low = 1 | Arthralgias in hands, wrists, ankles, and feet, accompanied |
| | | by morning stiffness (>30 minutes) |
| | Moderate = 2 | 1 to 5 (of 28 total count) synovitis |
| | High = 3 | ≥6 (of 28 total count) synovitis |
| Cutaneous [3] | No = 0 | Absence of currently active cutaneous involvement |
| Rate as “No activity” stable | Low = 1 | Erythema multiforme |
| long-lasting features related | Moderate = 2 | Limited cutaneous vasculitis, including urticarial vasculitis, |
| to damage | | or purpura limited to feet and ankle, or subacute cutaneous |
| | | lupus |
| | High = 3 | Diffuse cutaneous vasculitis, including urticarial vasculitis, |
| | | or diffuse purpura, or ulcers related to vasculitis |
| Pulmonary [5] | No = 0 | Absence of currently active pulmonary involvement |
| Rate as “No activity” stable | Low = 1 | Persistent cough or bronchial involvement with no |
| long-lasting features related | | radiographic abnormalities on radiography; or radiological |
| to damage, or respiratory | | or HRCT evidence of interstitial lung disease with: no |
| involvement not related to the | | breathlessness and normal lung function test |
| disease (tobacco use etc.) | Moderate = 2 | Moderately active pulmonary involvement, such as |
| | | interstitial lung disease shown by HRCT with shortness of |
| | | breath on exercise (NHYA II) or abnormal lung function |
| | | tests restricted to: 70% > DLCO ≥ 40% or 80% > FVC ≥ 60% |
| | High = 3 | Highly active pulmonary involvement, such as interstitial |
| | | lung disease shown by HRCT with shortness of breath at rest |
| | | (NHYA III, IV) or with abnormal lung function tests: 70% > |
| | | DLCO ≥ 40% or 80% > FVC ≥ 60% |
| Renal [5] | No = 0 | Absence of currently active renal involvement with |
| Rate as “No activity” stable | | proteinuria <0.5 g/day, no hematuria, no leucocyturia, no |
| long-lasting features related | | acidosis, or long-lasting stable proteinuria due to damage |
| to damage, and renal | Low = 1 | Evidence of mild active renal involvement, limited to tubular |
| involvement not related to the | | acidosis without renal failure or glomerular involvement |
| disease. If biopsy has been | | with proteinuria (between 0.5 and 1 g/day) and without |
| performed, please rate | | hematuria or renal failure (GFR ≥60 mL/min) |
| activity based on histological | Moderate = 2 | Moderately active renal involvement, such as tubular |
| features first | | acidosis with renal failure (GFR <60 mL/min) or glomerular |
| | | involvement with proteinuria between 1 and 1.5 g/day and |
| | | without hematuria or renal failure (GFR ≥60 mL/min) or |
| | | histological evidence of extra-membranous |
| | | glomerulonephritis or important interstitial lymphoid |
| | | infiltrate |
| | High = 3 | Highly active renal involvement, such as glomerular |
| | | involvement with proteinuria >1.5 g/day or hematuria or |
| | | renal failure (GFR <60 mL/min), or histological evidence of |
| | | proliferative glomerulonephritis or cryoglobulinemia related |
| | | renal involvement |
| Muscular [6] | No = 0 | Absence of currently active muscular involvement |
| Exclusion of weakness due to | Low = 1 | Mild active myositis shown by abnormal EMG or biopsy |
| corticosteroids | | with no weakness and creatine kinase (N < CK ≤ 2N) |
| | Moderate = 2 | Moderately active myositis proven by abnormal EMG or |
| | | biopsy with weakness (maximal deficit of 4/5), or elevated |
| | | creatine kinase (2N < CK ≤ 4N) |
| | High = 3 | Highly active myositis shown by abnormal EMG or biopsy |
| | | with weakness (deficit ≤3/5) or elevated creatine kinase |
| | | (>4N) |
| PNS [5] | No = 0 | Absence of currently active PNS involvement |
| Rate as “No activity” stable | Low = 1 | Mild active peripheral nervous system involvement, such as |
| long-lasting features related | | pure sensory axonal polyneuropathy shown by NCS or |
| to damage or PNS involvement | | trigeminal (V) neuralgia |
| not related to the disease | Moderate = 2 | Moderately active PNS involvement shown by NCS, such as |
| | | axonal sensory-motor neuropathy with maximal motor |
| | | deficit of 4/5, pure sensory neuropathy with presence of |
| | | cryoglobulinemic vasculitis, ganglionopathy with symptoms |
| | | restricted to mild/moderate ataxia, CIDP with mild |
| | | functional impairment (maximal motor deficit of 4/5 or mild |
| | | ataxia), or cranial nerve involvement of peripheral origin |
| | | (except trigeminal [V] neuralgia) |
| | High = 3 | Highly active PNS involvement shown by NCS, such as |
| | | axonal sensory-motor neuropathy with motor deficit ≤3/5, |
| | | peripheral nerve involvement due to vasculitis (mononeuritis |
| | | multiplex, etc.), severe ataxia due to ganglionopathy, CIDP |
| | | with severe functional impairment: motor deficit ≤3/5 or |
| | | severe ataxia |
| CNS [5] | No = 0 | Absence of currently active CNS involvement |
| Rate as “No activity” stable | Low = 1 | Moderately active CNS features, such as cranial nerve |
| long-lasting features related | | involvement of central origin, optic neuritis, or multiple |
| to damage or CNS involvement | | sclerosis-like syndrome with symptoms restricted to pure |
| not related to the disease | | sensory impairment or proven cognitive impairment |
| | High = 3 | Highly active CNS features, such as cerebral vasculitis with |
| | | cerebrovascular accident or transient ischemic attack, |
| | | seizures, transverse myelitis, lymphocytic meningitis, |
| | | multiple sclerosis-like syndrome with motor deficit |
| Hematological [2] | No = 0 | Absence of autoimmune cytopenia |
| For anemia, neutropenia, and | Low = 1 | Cytopenia of autoimmune origin with neutropenia |
| thrombopenia, only | | (1000 < neutrophils < 1500/mm³), and/or anemia |
| autoimmune cytopenia must | | (10 < hemoglobin < 12 g/dl), and/or thrombocytopenia |
| be considered | | (100 000 < platelets < 150 000/mm³); or lymphopenia (500 < |
| Exclusion of vitamin or iron | | lymphocytes < 1000/mm³) |
| deficiency, drug-induced | Moderate = 2 | Cytopenia of autoimmune origin with neutropenia |
| cytopenia | | (500 ≤ neutrophils ≤ 1000/mm³), and/or anemia |
| | | (8 ≤ hemoglobin ≤ 10 g/dl), and/or thrombocytopenia |
| | | (50 000 ≤ platelets ≤ 100 000/mm³); or lymphopenia |
| | | (≤500/mm³) |
| | High = 3 | Cytopenia of autoimmune origin with neutropenia |
| | | (neutrophils <500/mm³) and/or anemia (hemoglobin <8 g/dl) |
| | | and/or thrombocytopenia (platelets <50 000/mm³) |
| Biological [1] | No = 0 | Absence of any of the following biological features |
| | Low = 1 | Clonal component and/or hypocomplementemia (low C4 or |
| | | C3 or CH50) and/or hypergammaglobulinemia or high IgG |
| | | level between 16 and 20 g/L |
| | Moderate = 2 | Presence of cryoglobulinemia and/or |
| | | hypergammaglobulinemia or high IgG level >20 g/L, and/or |
| | | recent onset hypogammaglobulinemia or recent decrease of |
| | | IgG level (<5 g/L) |

CIDP = chronic inflammatory demyelinating polyneuropathy;
CK = creatine kinase;
CNS = central nervous system;
DLCO = diffusing capacity of the lungs for carbon monoxide;
EMG = electromyogram;
ESSDAI = EULAR Sjögren's syndrome disease activity index;
FVC = forced vital capacity;
GFR = glomerular filtration rate;
HRCT = high-resolution computed tomography;
IgG = immunoglobulin G;
N = normal;
NCS = nerve conduction studies;
NHYA = New York Heart Association (classification);
PNS = peripheral nervous system

### ClinESSDAI

ClinESSDAI derives from the ESSDAI, and its score provides an accurate evaluation of disease activity independent of B-cell biomarkers. The clinical domains in clinESSDAI have different weights than in ESSDAI (Table S5).

TABLE S5
Comparison of Domain Weights of
Original ESSDAI and clinESSDAI

| Domain (activity level) | ESSDAI | clinESSDAI |
|---|---|---|
| Constitutional (0-2) | 3 | 4 |
| Lymphadenopathy (0-3) | 4 | 4 |
| Glandular (0-2) | 2 | 2 |
| Articular (0-3) | 2 | 3 |
| Cutaneous (0-3) | 3 | 3 |
| Pulmonary (0-3) | 5 | 6 |
| Renal (0-3) | 5 | 6 |
| Muscular (0-3) | 6 | 7 |
| Peripheral nervous system (0-3) | 5 | 5 |
| Central nervous system (0-3) | 5 | 5 |
| Hematological (0-3) | 2 | 2 |
| Biological (0-2) | 1 | NA |
| Score total | 0-123 | 0-135 |

clinESSDAI = clinical EULAR Sjögren's syndrome disease activity index;
ESSDAI = EULAR Sjögren's syndrome disease activity index;
EULAR = European Alliance of Associations for Rheumatology;
NA = not applicable

### STAR

STAR was developed to assess the efficacy of treatments for pSS. A secondary efficacy endpoint for this study is the proportion of responders (STAR score of ≥5) at week 24.

This composite measure contains 5 domains:
Systemic activity: 3 points
   clinESSDAI decrease of ≥3 points;
Patient-reported outcome: 3 points
   ESSPRI decrease of at least 1 point or ≥15% (see below)
   Symptoms of dryness, pain, and fatigue rated on 3 numeric rating scales;
Lacrimal gland function (assessed by Schirmer's test or OSS): 1 point
   Schirmer's test (see below)
      If abnormal score at baseline: increase of ≥5 mm from baseline;
      If normal score at baseline: no change to abnormal;
   OSS (see below)
      If abnormal score at baseline: decrease of ≥2 points from baseline;
      If normal score at baseline: no change to abnormal;
Salivary gland function: 1 point
   UWSF:
      If score >0 at baseline: increase of ≥25% from baseline;
      If score is 0 at baseline: any increase in UWSF from baseline;
OR
   SGUS:
      ≥25% decrease in total Hocevar score from baseline;
Biological (assessed by IgG or RF): 1 point
   IgG: ≥10% reduction;
   RF: ≥25% decrease.

### Patient-Reported Outcomes

### ESSPRI

ESSPRI is a questionnaire developed to measure self-reported symptoms in participants with pSS. The ESSPRI has 3 items that measure dryness, fatigue, and pain over a recall period of “the last 2 weeks.” Each item includes a numeric rating scale ranging from 0 “No symptoms (dryness, fatigue or pain)” to 10 “Maximal imaginable (dryness, fatigue or pain).” The total global score ranges from 0 to 10 and the ESSPRI is calculated by averaging the numeric scores for pain, fatigue, and dryness, with higher scores indicating more symptoms. It has been shown to correlate well with PGA and has been validated in participants with pSS.

### MFI

The MFI is a 20-item scale designed to evaluate 5 dimensions of fatigue: general fatigue, physical fatigue, reduced motivation, reduced activity, and mental fatigue. Participants are asked to report their fatigue over a recall period of “lately” (past 7 days). Each item in the MFI includes 5 boxes ranging from “yes, that is true” to “no, that is not true,” and participants are asked to indicate how true certain statements are in regard to their experience with fatigue. Scores in each domain range from 4 to 20, with lower scores corresponding to better health status.

### PGA

PGA is a tool that measures a participant's global evaluation of their overall disease activity at the time of assessment.

The participant rates their overall disease activity by drawing a vertical mark on a 10-cm VAS from the left end of the line (no evidence of disease activity) to the right end of the line (extremely active or severe disease activity).

### SF-36

The SF-36 is a 36-item scale constructed to survey health-related quality of life on 8 domains: limitations in physical activities because of health problems; limitations in social activities because of physical or emotional problems; limitations in usual role activities because of physical health problems; bodily pain; general mental health (psychological distress and well-being); limitations in usual role activities because of emotional problems; vitality (energy and fatigue); and general health perceptions. The SF-36 includes Yes/No questions in addition to 3 point, 5 point, or 6 point Likert response scales. Participants are required to complete the SF-36 using the recall period of “the last 4 weeks.” SF-36 scores are calculated by domain, and total calculated scores for each domain can range from 0 to 100, with higher scores being equated to better health status. The SF-36 is scored into 2 summary scores: physical component summary (consisting of physical function, physical role, bodily pain, and general health domain total scores) and mental component summary (consisting of vitality, mental health, role emotional, and social function domain total scores).

### PASS

PASS is a patient-reported outcome measure that assesses the “value beyond which patients consider themselves well.” PASS measures participant well-being and overall feeling that symptoms are in remission, through a single question that is dependent on the indication.

PASS assesses the level of symptoms at which participants with rheumatic diseases consider themselves well. To record the PASS, the rheumatologist asks the participant whether they consider their current state to be satisfactory, considering all of the consequences of their disease.

### EO-5D-5L

EQ-5D-5L is a standardized measure of health status. It was developed by the EuroQol Group to provide a simple, generic measure of health status for clinical and economic appraisal. The descriptive system comprises 5 dimensions:
Mobility;
Self-care;
Usual activities;
Pain/discomfort;
Anxiety/depression.

Each dimension has 5 levels:
No problem;
Slight problem;
Moderate problem;
Severe problem;
Extreme problem.
The participant is asked to indicate their health state by selecting the box next to the most appropriate statement in each of the 5 dimensions using the recall period “today.” This decision results in a 1-digit number expressing the level selected for that dimension. The digits for 5 dimensions were combined in a 5-digit number describing the respondent's health state. A unique health state is defined by combining 1 level from each of the 5 dimensions. A total of 3125 possible health states could be defined in this way. Each state is referred to in terms of a 5-digit code. For example, state 11111 would indicate no problems in any of the 5 dimensions, and 12345 would indicate no problem with mobility, slight problems with washing or dressing, moderate problems with doing usual activities, severe pain or discomfort, and extreme anxiety or depression.
A VAS is included in the questionnaire. Respondents are asked to mark the health status from 0 to 100 on the day the interview is conducted, with a score of 0 corresponding to “the worst health you can imagine” and 100 corresponding to “the best health you can imagine.”

### Additional Efficacy Measures

### SGUS

The SGUS grading system of Hocevar et al. rates 5 parameters: parenchymal echogenicity, homogeneity, presence of hypoechogenic areas, hypoechogenic reflections, and the clearness of salivary gland borders. The overall ultrasound score is calculated by summation of the grades for the 5 subscores for all 4 major salivary glands. The overall ultrasound score can range from 0 to 48.

### Salivary Flow Rate

SWSF and UWSF rates are assessed in this study.

### Schirmer's Test

Schirmer's test is an assessment of tear gland function in which a strip of filter paper is applied under the eyelid to measure the quantity of tear production. A result of ≤5 mm indicates abnormal tear gland function.

### OSS

OSS is used in this study to assess tear gland function in participants with pSS. OSS uses lissamine green dye to grade the conjunctiva, and fluorescent dye to grade the cornea. A score of ≥3 points indicates abnormal tear gland function.

### E. Pharmacokinetics

Blood samples for PK analysis are collected predose on IMP administration visits (preferably within the 2 hours before IMP infusion) and within the 30 minutes after the end of the infusion as described in Table S3. Efgartigimod serum concentrations are determined using a validated method.

### F. Pharmacodynamics

Baseline and postbaseline PD blood samples are collected predose, preferably within 2 hours before IMP administration at the time points described in Table S3.

Total IgG levels are determined using validated methods at a central laboratory. IgG is assessed at screening as part of the eligibility criteria and secondary efficacy measures.

Participants are also tested for anti-Ro/SS A and anti-La/SS-B autoantibodies at the time points described in Table S3, as a part of inclusion criteria requirements and secondary efficacy measures.

### G. Biomarkers

Blood is collected, whereas serum and PBMCs are aliquoted at the time points described in Table S3 to explore the relation between relevant biomarkers and clinical effects.

PBMCs are used for immunophenotyping using flow cytometry, including but not limited to the assessment of the proportion of B cells within the CD45+ population. In addition, these samples may be analyzed for changes in gene expression. Gene expression analysis is conducted using RNA sequencing for genes relevant to participant immune status and related pathways, including the IFN pathway, to understand participant response to efgartigimod therapy. This is performed only if both efficacy and biomarker data support the hypothesis. Testing is optional if it is categorized as genetic testing by local regulations.

Serum is used for the analysis of autoantibodies, immune complexes, quantification of chemokines/cytokines, and markers of complement activation (including but not limited to C3, C4, and/or split products thereof).

Saliva is collected at the time points described in Table S3 to explore the relation between salivary proteins and clinical effects.

### H. Immunogenicity Assessments

Blood samples are collected predose (within the 2 hours before IMP infusion) at the time points described in Table S3 to evaluate the serum levels of ADAs against efgartigimod.

Samples are analyzed by the designated laboratory in a tiered approach using validated immunogenicity assays.

### I. Objectives and Endpoints

TABLE S6
Objectives and endpoints

| | |
|---|---|
| Objectives | Endpoints |

Primary

| | |
|---|---|
| To evaluate the effect of | Proportion of CRESS responders on >3 of 5 items at week 24. The 5 |
| efgartigimod IV compared to | items are: |
| placebo on CRESS | Systemic disease activity: clinESSDAI |
| | Patient-reported symptoms: ESSPRI |
| | Tear gland function: Schirmer's test and OSS |
| | Salivary gland function: UWSF rate and SGUS |
| | Serology (serum IgG and/or RF) |

Secondary

| | |
|---|---|
| To evaluate the effect of | Change in the relative counts of lymphocytic infiltrate (stained for |
| efgartigimod IV compared to | CD45) at week 24 |
| placebo on the histology of | Change in B/B + T cell ratio at week 24 |
| the parotid gland | |
| To evaluate the safety of | Incidence and severity of TEAEs, AESIs, and SAEs by SOC and PT |
| efgartigimod IV compared to | Changes in vital sign measurements, ECG results, and clinical |
| placebo in participants with | laboratory safety evaluations |
| pSS | |
| To evaluate the effect of | Proportion of participants with minimal clinically important |
| efgartigimod IV compared to | improvement in ESSDAI: improvement of ≥3 points in ESSDAI |
| placebo on clinical efficacy | score at week 24 |
| parameters | Proportion of participants with low disease activity: ESSDAI score |
| | of <5 at week 24 |
| | Proportion of participants with minimal clinically important |
| | improvement in clinESSDAI: improvement of ≥3 points in |
| | clinESSDAI score at week 24 |
| | Proportion of participants with low disease activity: clinESSDAI |
| | score of <5 at week 24 |
| | Proportion of participants with minimal clinically important |
| | improvement in ESSPRI: decrease of 1 point or ≥15% at week 24 |
| | Change in ESSDAI score at week 24 |
| | Change in clinESSDAI score at week 24 |
| | Change in ESSPRI score at week 24 |
| To evaluate the effect of | Proportion of participants with STAR score of ≥5 at week 24 |
| efgartigimod IV compared to | |
| placebo on STAR | |
| To evaluate the PK of | Efgartigimod serum concentration-time profile |
| efgartigimod IV | |
| To evaluate the PD of | Values, changes from baseline, and percent reduction from baseline |
| efgartigimod IV | in total IgG levels in serum |
| | Values, changes from baseline, and percent reduction from baseline |
| | in autoantibodies in serum: |
| | Anti-Ro/SS-A |
| | Anti-La/SS-B |
| To evaluate the | Incidence and prevalence of ADA against efgartigimod in serum |
| immunogenicity of | |
| efgartigimod IV | |

Exploratory

| | |
|---|---|
| To explore the effect of | Proportion of responders on 2 of 4 items of the adjusted CRESS |
| efgartigimod IV compared to | (without serology item) at week 24 |
| placebo on additional | Change in activity of individual ESSDAI domains at week 24 |
| clinical pSS measures | |
| To explore the effect of | Change in SWSF rate at week 24 |
| efgartigimod IV compared to | Change in UWSF rate at week 24 |
| placebo on glandular | Change in Hocevar score at week 24 |
| measures | Change in Schirmer's test at week 24 |
| | Change in OSS at week 24 |
| | Response in salivary gland as defined in CRESS at week 24 |
| | Response in tear gland as defined in CRESS at week 24 |
| To explore the effect of | Change in total MFI score at week 24 |
| efgartigimod IV compared to | Change in physical component and mental component scores of |
| placebo on patient-reported | SF-36 at week 24 |
| outcomes | Change in PGA at week 24 |
| | Change in individual ESSPRI scores at week 24 |
| | Change in EQ-5D-5L utilities score at week 24 |
| | Change in VAS score at week 24 |
| | PASS at week 24 |
| To explore the effect of | Changes in immunophenotype, focus score, number of germinal |
| efgartigimod IV compared to | centers, lymphoepithelial lesions, and gene expression per mm² in |
| placebo on additional | parotid gland parenchyma at week 24 |
| histological measures on | |
| parotid gland biopsy | |
| To explore the effect of | Change in cytokine/chemokine profiles (including but not limited to |
| efgartigimod IV compared to | BAFF, type 1 interferon [IFN]) at weeks 4, 16, and 24 |
| placebo on serum | Reduction in C1q immune complexes at weeks 4, 16, and 24 |
| biomarkers | Reduction in IgA, IgM, and IgG-related autoantibodies at weeks 4, |
| | 16, and 24 |
| | Values, changes from baseline, and percent reduction from baseline |
| | in RF in serum |
| | Reduction in serum markers of complement activation (including but |
| | not limited to C3, C4, and/or split products thereof) at weeks 4, 16, |
| | and 24 |
| To explore the effect of | Change in immunophenotyping in peripheral blood using flow |
| efgartigimod IV compared to | cytometry at weeks 4 and 24 |
| placebo on blood biomarkers | Change in gene expression profile (RNA sequencing) at weeks 4, 16, |
| | and 24 |
| To explore the effect of | Change in salivary proteins at weeks 4, 16, and 24 |
| efgartigimod IV compared to | |
| placebo on saliva biomarkers | |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A method of treating primary Sjögren's syndrome (pSS) in a subject in need thereof, the method comprising administering to the subject an effective amount of a human neonatal Fc receptor (FcRn) antagonist, wherein the FcRn antagonist comprises or consists of a variant Fc region or FcRn binding fragment thereof.

2-5. (canceled)

6. The method of claim 1, wherein the variant Fc region comprises or consists of a first Fc domain and a second Fc domain which form a homodimer or heterodimer, wherein the first Fc domain and/or the second Fc domain comprise amino acids Y, T, E, K, and F at EU positions 252, 254, 256, 433, and 434, respectively.

7. (canceled)

8. The method of claim 6, wherein the first Fc domain and/or the second Fc domain comprise amino acids Y, T, E, K, F, and Y at EU positions 252, 254, 256, 433, 434, and 436, respectively.

9. The method of claim 6, wherein the first Fc domain and/or the second Fc domain comprise an amino acid sequence independently selected from the group consisting of SEQ ID NO: 2, SEQ ID NO: 3, SEQ ID NO: 20, and SEQ ID NO: 21.

10. (canceled)

11. The method of claim 1, wherein the FcRn antagonist is efgartigimod.

12-13. (canceled)

14. The method of claim 1, wherein the FcRn antagonist is administered intravenously once weekly or once every two weeks.

15-16. (canceled)

17. The method of claim 1, wherein the FcRn antagonist is administered intravenously at a dose of 10 mg/kg to 30 mg/kg once weekly or once every two weeks.

18. The method of claim 1, wherein the FcRn antagonist is administered intravenously at a dose of 10 mg/kg once weekly or once every two weeks.

19. (canceled)

20. The method of claim 1, wherein the FcRn antagonist is administered subcutaneously once weekly, once every two weeks, once every three weeks, once every four weeks, once monthly, or once every six weeks.

21. (canceled)

22. The method of claim 20, wherein the FcRn antagonist is administered subcutaneously at a fixed dose of 750 mg to 3000 mg once weekly, once every two weeks, once every three weeks, once every four weeks, once monthly, or once every six weeks.

23-27. (canceled)

28. The method of claim 1, further comprising administering to the subject an effective amount of one or more of a corticosteroid, an antimalarial, a disease-modifying anti-rheumatic drug (DMARD), a janus kinase (JAK) inhibitor, a pharmacological stimulant for salivary and lacrimal glands, an anticholinergic agent, or a topical ophthalmic agent.

29. The method of claim 28, wherein the corticosteroid is a systemic corticosteroid or a topical corticosteroid.

30. (canceled)

31. The method of claim 1, wherein the subject:
a) meets the ACR-EULAR classification criteria for pSS;
b) met the ACR-EULAR classification ≤7 years before administration of the FcRn antagonist;
c) has at least a moderate level of systemic disease activity;
d) has a EULAR Sjögren's syndrome disease activity index (ESSDAI) score of ≥5;
e) has a detectable serum level of a pSS-related autoantibody;
f) has a detectable serum level of an anti-Ro/SS-A antibody or an anti-La/SS-B antibody: and/or
g) has an unstimulated whole salivary flow (UWSF) rate>0 and/or a stimulated whole salivary flow (SWSF) rate>0.10.

32-37. (canceled)

38. The method of claim 1, wherein the subject shows one or more responses following administration of the FcRn antagonist, wherein the responses are selected from the group consisting of:
a) a clinical ESSDAI (clinESSDAI) score of <5 points;
b) a decrease in EULAR Sjögren's syndrome patient reported index (ESSPRI) score of ≥1 point or ≥15%, compared to a baseline value;
c) an increase in tear gland function;
d) an increase in salivary gland function; and
e) a decrease in serum rheumatoid factor (RF) of at least 25%, compared to a baseline value, or a decrease in serum IgG of at least 10%, compared to a baseline value.

39-42. (canceled)

43. The method of claim 1, wherein the subject shows a change in CD45+lymphocytic infiltrate in the parotid gland following administration of the FcRn antagonist, compared to a baseline value.

44-45. (canceled)

46. The method of claim 1, wherein the subject shows a change in B/B+T cell ratio in the parotid gland following administration of the FcRn antagonist, compared to a baseline value.

47-48. (canceled)

49. The method of claim 1, wherein the subject shows a decrease in ESSDAI score, clinESSDAI score, and/or ESSPRI score following administration of the FcRn antagonist, compared to a baseline value.

50-53. (canceled)

54. The method of claim 1, wherein the subject shows an increase in Sjögren's Tool for Assessing Response (STAR) score following administration of the FcRn antagonist, compared to a baseline value.

55-56. (canceled)

57. The method of claim 1, wherein the subject shows an improvement in total Multidimensional Fatigue Inventory (MFI) score, SF-36 physical component score, SF-36 mental component score, PGA score, EQ-5D-5L score, VAS score, ESSPRI dryness score, ESSPRI fatigue score, ESSPRI pain score, and/or PASS score, following administration of the FcRn antagonist, compared to a baseline value.

58. (canceled)

59. The method of claim 1, wherein the subject shows a change in SWSF rate, UWSF rate, Hocevar score, Schirmer's test score, and/or OSS, following administration of the FcRn antagonist, compared to a baseline value.

60. (canceled)

61. The method of claim 1, wherein the subject shows a reduction in a serum level of total IgG, RF, an autoantibody, a cytokine/chemokine, an immune complex, or a marker of complement activation following administration of the FcRn antagonist, compared to a baseline value.

62-78. (canceled)


SEQUENCE LISTING (33 sequences)

SEQ ID NO: 1 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 5 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 6 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 7 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 8 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 10 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 11 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 12 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 13 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 14 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 15 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 16 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 17 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 18 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 19 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 20 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 21 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 22 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 23 - protein, 216 aa, synthetic construct
QSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 24 - protein, 445 aa, synthetic construct
EVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 219 aa, synthetic construct
DIQMTQSPSSLSASVGDRVTITCKSSQSLVGASGKTYLYWLFQKPGKAPKRLIYLVSTLDSGIPSRFSGSGSGTEFTLTISSLQPEDFATYYCLQGTHFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, synthetic construct
EVPLVESGGGLVQPGGSLRLSCAVSGFTFSNYGMVWVRQAPGKGLEWVAYIDSDGDNTYYRDSVKGRFTISRDNAKSSLYLQMNSLRAEDTAVYYCTTGIVRPFLYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 214 aa, synthetic construct
DIQMTQSPSSLSASVGDRVTITCKASDHINNWLAWYQQKPGQAPRLLISGATSLETGVPSRFSGSGTGKDYTLTISSLQPEDFATYYCQQYWSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 444 aa, synthetic construct
QVQLVQSGAELKKPGASVKLSCKASGYTFTSYGISWVKQATGQGLEWIGEIYPRSGNTYYNEKFKGRATLTADKSTSTAYMELRSLRSEDSAVYFCARSTTVRPPGIWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 214 aa, synthetic construct
SYVLTQSPSVSVAPGQTARITCGGNNIGSKSVHWYQQKPGQAPVLVVYDDSDRPSGIPERFSASNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 450 aa, synthetic construct
QLLLQESGPGLVKPSETLSLTCTVSGGSLSSSFSYWVWIRQPPGKGLEWIGTIYYSGNTYYNPSLKSRLTISVDTSKNHFSLKLSSVTAADTAVYYCARRAGILTGYLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 15 aa, synthetic construct
EPKSCDKTHTCPPCP

SEQ ID NO: 32 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 33 - protein, 447 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCPNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY